(19) United States
(12) Patent Application Publication
(10) Pub. No.: US 2025/0177409 A1
(43) Pub. Date: Jun. 5, 2025

(54) COMBINATION THERAPY OF PI3K INHIBITOR AND PD-1 INHIBITOR

(71) Applicant: MEI Pharma, Inc., San Diego, CA (US)

(72) Inventors: Daniel P. GOLD, San Diego, CA (US); Richard GHALIE, San Diego, CA (US)

(21) Appl. No.: 18/841,989

(22) PCT Filed: Feb. 28, 2023
(86) PCT No.: PCT/US2023/014114

§ 371 Date: Aug. 27, 2024

Related U.S. Application Data
(60) Provisional application No. 63/315,380, filed on Mar. 1, 2022.

(51) Int. Cl. A61K 31/5377; A61K 39/00; A61P 35/00; C07K 16/28

(52) U.S. Cl. CPC A61K 31/5377; A61P 35/00; C07K 16/2818; A61K 2039/505; A61K 2039/545

(57) ABSTRACT

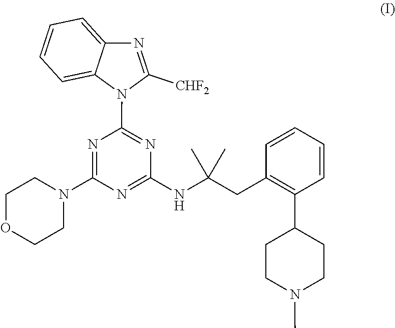 [SMILES: CN1CCC(c2ccccc2CC(C)(C)Nc2nc(N3CCOCC3)nc(-n3c(C(F)F)nc4ccccc43)n2)CC1]

Provided herein are methods of treating proliferative diseases or disorders (e.g., a cancer) in a patient in need thereof using a combination therapy of Compound (I) or a pharmaceutically acceptable salt thereof and a PD-1 inhibitor (e.g., Pembrolizumab).

### CROSS-REFERENCE

This International Patent Application claims the benefit of U.S. Provisional Patent Application No. 63/315,380, filed Mar. 1, 2022, which is incorporated herein by reference in its entirety.

### BACKGROUND OF THE INVENTION

Phosphoinositide-3-kinases (PI3Ks) play a variety of roles in normal tissue physiology (Foukas & Shepherd, Biochem. Soc. Trans. 2004, 32, 330; Shepherd, Acta Physiol. Scand. 2005, 183, 3), with p110α having a specific role in cancer growth, p110β in thrombus formation mediated by integrin α_{π}β₃ (Jackson et al., Nat. Med. 2005, 11, 507), and p110γ, in inflammation, rheumatoid arthritis, and other chronic inflammation states (Barber et al., Nat. Med. 2005, 11, 933; Camps et al., Nat. Med. 2005, 11, 936; Rommel et al., Nat. Rev. 2007, 7, 191; and Ito, et al., J. Pharm. Exp. Therap. 2007, 321, 1). Inhibitors of PI3Ks have therapeutic potential in the treatment of various proliferative diseases, including cancer.

### SUMMARY OF THE DISCLOSURE INVENTION

Provided herein are methods for treating a cancer in a patient in need thereof in one or more treatment cycles, comprising:
(i) administering Compound I or a pharmaceutically acceptable salt thereof to the patient for a first period of time within each of the one or more treatment cycles; and
(ii) administering a PD-1 inhibitor to the patient after the administration of Compound I or a pharmaceutically acceptable salt thereof,
wherein Compound I has the structure of:

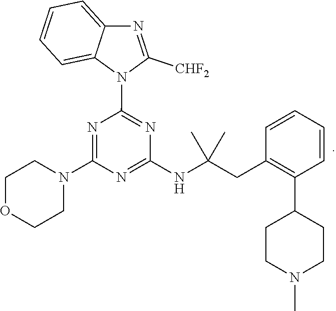 [SMILES: CN1CCC(c2ccccc2CC(C)(C)Nc2nc(N3CCOCC3)nc(-n3c(C(F)F)nc4ccccc43)n2)CC1]

In some embodiments, each of the one or more treatment cycles is at least about 3, 4, 5, 6, 7, 8, 9, 10, 11, or 12 weeks.

In some embodiments, each of the one or more treatment cycles is about 6 weeks.

In some embodiments, each of the one or more treatment cycles is about 3 weeks.

In some embodiments, the PD-1 inhibitor is administered about 7, 8, 9, 10, 11, 12, 13, 14, 15, 16, 17, 18, 19, or 20 days after the first period of time.

In some embodiments, each of the one or more treatment cycles is about 6 weeks and the PD-1 inhibitor is administered about 16 days after the first period of time.

In some embodiments, each of the one or more treatment cycles is about 3 weeks and the PD-1 inhibitor is administered about 8 days after the first period of time.

In some embodiments, the first period of time is 3 days, 4 days, 5 days, 6 days, or 1 week.

In some embodiments, the first period of time is 5 days.

In some embodiments, after the first period of time, the PD-1 inhibitor is administered starting on the next day after a second period of time, wherein the second period of time is immediately after the first period.

In some embodiments, the second period of time is about 0, 1, 2, 3, 4, 5, 6, 7, 8, 9, 10, 11, 12, 13, 14, 15, 16, 17, 18, 19, or 20 days.

In some embodiments, the second period of time is about 16 days.

In some embodiments, the second period of time is about 8 days.

In some embodiments, Compound I or a pharmaceutically acceptable salt thereof is administered daily in the first period of time.

In some embodiments, Compound I or a pharmaceutically acceptable salt thereof is administered to the patient on an intermittent dosing schedule.

In some embodiments, Compound I or a pharmaceutically acceptable salt thereof is only administered for the first period of time of each of the one or more treatment cycles and not on the days after the first period of time, and the first period of time is shorter than each of the one or more treatment cycles.

In some embodiments, the first period of time is 3 days, 4 days, 5 days, 6 days, or 1 week.

In some embodiments, the first period of time is 5 days.

In some embodiments, Compound I or a pharmaceutically acceptable salt thereof is administered at a dosage of 60 mg/day.

In some embodiments, Compound I or a pharmaceutically acceptable salt thereof is administered orally.

In some embodiments, the PD-1 inhibitor is administered once, twice, or three times in each of the one or more treatment cycles.

In some embodiments, the PD-1 inhibitor is administered once in each of the one or more treatment cycles.

In some embodiments, when each of the one or more treatment cycles is about 6 weeks, the PD-1 inhibitor is administered on Day 22 of each of the one or more treatment cycles.

In some embodiments, when each of the one or more treatment cycles is about 3 weeks, the PD-1 inhibitor is administered on Day 14 of each of the one or more treatment cycles.

In some embodiments, the PD-1 inhibitor is administered twice in each of the one or more treatment cycles.

In some embodiments, when each of the one or more treatment cycles is about 6 weeks, the PD-1 inhibitor is administered on Day 14 and Day 35 of each of the one or more treatment cycles.

In some embodiments, the PD-1 inhibitor is pembrolizumab, nivolumab, cemiplimab, or dostarlimab, or a variant or biosimilar thereof, or combinations thereof.

In some embodiments, the PD-1 inhibitor is pembrolizumab, or a variant or biosimilar thereof.

In some embodiments, the PD-1 inhibitor is administered for at least 6 weeks.

In some embodiments, the PD-1 inhibitor is administered for 6, 9, 12, 15, 18, 21, 24, 27, or 30 weeks.

In some embodiments, the PD-1 inhibitor is administered once every 3 weeks or once every 6 weeks.

In some embodiments, the PD-1 inhibitor is administered at a dosage of 200 mg or 400 mg.

In some embodiments, the PD-1 inhibitor is administered once every 6 weeks.

In some embodiments, the PD-1 inhibitor is administered at a dosage of 400 mg.

In some embodiments, the PD-1 inhibitor is administered once every 3 weeks.

In some embodiments, the PD-1 inhibitor is administered at a dosage of 200 mg.

In some embodiments, the PD-1 inhibitor is administered intravenously.

In some embodiments, the first period starts on Day 1, Day 2, Day 3, Day 4, Day 5, Day 6, Day 7, or Day 8 of each of the one or more treatment cycles.

In some embodiments, the first period starts on Day 1 of each of the one or more treatment cycles.

In some embodiments, each of the one or more treatment cycles is 6 weeks, Compound I or a pharmaceutically acceptable salt thereof is administered on Days 1 to 5, and then the PD-1 inhibitor is administered once on Day 22.

In some embodiments, each of the one or more treatment cycles is 3 weeks, Compound I or a pharmaceutically acceptable salt thereof is administered on Days 1 to 5, and then the PD-1 inhibitor is administered once on Day 14.

In some embodiments, the cancer is lung cancer (e.g., non-small cell lung cancer), melanoma, renal cell cancer, head and neck cancer, breast cancer (e.g., triple-negative breast cancer), non-Hodgkin's lymphoma, Hodgkin lymphoma, colorectal cancer, urothelial carcinoma, gastric cancer, cervical cancer, hepatocellular carcinoma, Merkel cell carcinoma, esophageal cancer, endometrial cancer, cutaneous squamous cell carcinoma, or mesothelioma.

In some embodiments, the cancer is lung cancer.

In some embodiments, the lung cancer is non-small cell lung cancer.

In some embodiments, the cancer is melanoma.

In some embodiments, the cancer is renal cell cancer.

In some embodiments, the cancer is hepatocellular carcinoma.

In some embodiments, the cancer is colorectal cancer.

In some embodiments, the colorectal cancer is microsatellite instability-high or mismatch repair deficient colorectal cancer.

In some embodiments, the methods provided herein further comprising (iii) evaluating efficacy of the administration of the combination of Compound I or a pharmaceutically acceptable salt thereof and the PD-1 inhibitor by measuring levels of regulatory T-cells (CD25hi FoxP3+ T cells) or effector T cells (e.g., CD8+ and CD4+ cells).

In some embodiments, a level of regulatory T-cells (CD25hi FoxP3+ T cells) or effector T cells (e.g., CD8+ and CD4+ cells) that is less than or equal to about 70%, 65%, 60%, 55%, 50%, 45%, 40%, 35%, 30%, 25%, 20%, 15%, or 10% of a baseline level of the regulatory T-cells (CD25hi FoxP3+ T cells) or effector T cells (e.g., CD8+ and CD4+ cells) indicates that the one or more treatment cycles of administration of the administration of the combination of Compound I or a pharmaceutically acceptable salt thereof and the PD-1 inhibitor is effective.

In some embodiments, a level of regulatory T-cells (CD25hi FoxP3+ T cells) or effector T cells (e.g., CD8+ and CD4+ cells) that is less than or equal to about 65% of a baseline level of the regulatory T-cells (CD25hi FoxP3+ T cells) or effector T cells (e.g., CD8+ and CD4+ cells) indicates that the one or more treatment cycles of administration of the administration of the combination of Compound I or a pharmaceutically acceptable salt thereof and the PD-1 inhibitor is effective.

In some embodiments, the co-administration of Compound I or pharmaceutically acceptable salt thereof and the PD-1 inhibitor is effective in treating the cancer.

In another aspect, provided herein are methods for treating a cancer in a patient in need thereof in one or more treatment cycles, comprising: (i) administering Compound I or a pharmaceutically acceptable salt thereof to the patient on Days 1 to 5 of each of the one or more treatment cycles; and (ii) administering a PD-1 inhibitor to the patient on Day 22 of each of the treatment cycles; wherein each of the one or more treatment cycles is 6 weeks; and wherein Compound I has the structure of:

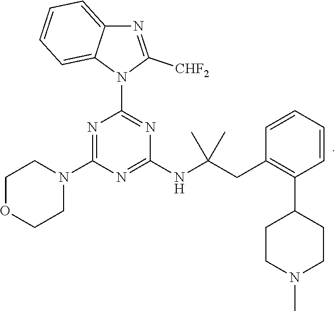 [SMILES: CN1CCC(c2ccccc2CC(C)(C)Nc2nc(N3CCOCC3)nc(-n3c(C(F)F)nc4ccccc43)n2)CC1]

In another aspect, provided herein are methods for treating a cancer in a patient in need thereof in one or more treatment cycles, comprising: (i) administering Compound I or a pharmaceutically acceptable salt thereof to the patient on Days 1 to 5 of each of the one or more treatment cycles; (ii) administering a PD-1 inhibitor to the patient on Day 14 of each of the treatment cycles; wherein each of the one or more treatment cycles is 3 weeks; and wherein Compound I has the structure of:

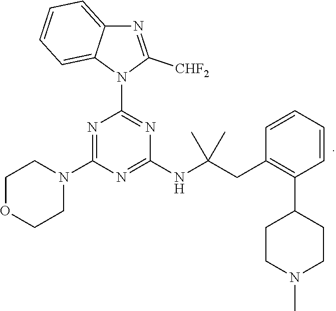 [SMILES: CN1CCC(c2ccccc2CC(C)(C)Nc2nc(N3CCOCC3)nc(-n3c(C(F)F)nc4ccccc43)n2)CC1]

In yet another aspect, provided herein are methods for treating a cancer in a patient in need thereof in one or more treatment cycles, comprising: (i) administering Compound I or a pharmaceutically acceptable salt thereof to the patient in Week 1 of odd cycles (e.g., Cycle 1) of the one or more treatment cycles; and (ii) administering a PD-1 inhibitor to the patient 1 in Week 1 of even cycles (e.g., Cycle 2) of each of the treatment cycles; wherein each of the one or more treatment cycles is 3, 6, or 9 weeks; and wherein Compound I has the structure of:

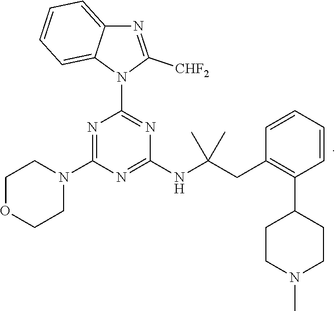 [SMILES: CN1CCC(c2ccccc2CC(C)(C)Nc2nc(N3CCOCC3)nc(-n3c(C(F)F)nc4ccccc43)n2)CC1]

In yet another aspect, provided herein are methods for treating a cancer in a patient in need thereof in one or more treatment cycles, comprising: (i) administering Compound I or a pharmaceutically acceptable salt thereof to the patient in Week 1 of each of the one or more treatment cycles; and (ii) administering a PD-1 inhibitor to the patient in Week 2 of each of the treatment cycles; wherein each of the one or more treatment cycles is 3 weeks; and wherein Compound I has the structure of:

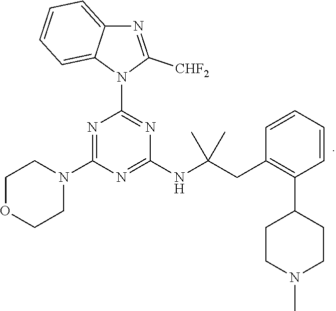 [SMILES: CN1CCC(c2ccccc2CC(C)(C)Nc2nc(N3CCOCC3)nc(-n3c(C(F)F)nc4ccccc43)n2)CC1]

In yet another aspect, provided herein are methods for treating a cancer in a patient in need thereof, comprising: (i) administering Compound I or a pharmaceutically acceptable salt thereof to the patient for 1, 2, 3, 4, or 5 days once daily in every 3, 6, or 9 weeks; and (ii) administering a PD-1 inhibitor to the patient once every 3 or 6 weeks; wherein Compound I has the structure of:

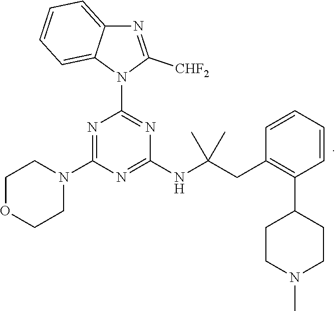 [SMILES: CN1CCC(c2ccccc2CC(C)(C)Nc2nc(N3CCOCC3)nc(-n3c(C(F)F)nc4ccccc43)n2)CC1]

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the invention are utilized, and the accompanying drawings of which:

FIG. 1 illustrates the Kaplan-Meier survival curves obtained from Example 2a, wherein a study was performed evaluating the efficacy of a combination treatment of Compound I and a PD-1 inhibitor (RMP1-14), in a MC38 murine adenocarcinoma model. The figure depicts the efficacy of two treatment cycles of the combination treatment in comparison to a single cycle of a combination treatment or a single cycle of the PD-1 inhibitor.

FIG. 2 illustrates the Kaplan-Meier survival curves obtained from Example 2a, wherein a study was performed evaluating the efficacy of a combination treatment of Compound I and the PD-1 inhibitor (RMP1-14) in a MC38 murine adenocarcinoma model. The figure depicts the efficacy of two treatment cycles of the combination treatment in comparison to a single cycle of a combination treatment or a single cycle of the combination with an additional cycle of Compound I.

FIG. 3 illustrates the Kaplan-Meier survival curves obtained from Example 2a, wherein a study was performed evaluating the efficacy of a combination treatment of Compound I and the PD-1 inhibitor (RMP1-14) in a MC38 murine adenocarcinoma model. The figure depicts the efficacy of two treatment cycles of the combination treatment in comparison to a single cycle of a combination treatment or a single cycle of the combination with an additional cycle of the PD-1 inhibitor.

FIG. 4 depicts the study design for the clinical study of Compound I in combination with Pembrolizumab for treating certain cancer patients.

FIG. 5 illustrates the dosing schedule of level 1 (top) and level 2 (bottom) of the study design Compound I in combination with Pembrolizumab.

### DETAILED DESCRIPTION OF THE INVENTION

Provided herein are methods of treating proliferative diseases and disorders (e.g., cancers) in a patient in need thereof in one or more treatment cycles, in each treatment cycle, comprising administering i) a PI3K inhibitor that is Compound I or a pharmaceutically acceptable salt thereof; and ii) a PD-1 inhibitor (e.g., pembrolizumab).

### Definitions

To facilitate understanding of the disclosure set forth herein, a number of terms are defined below.

Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject, in one embodiment, a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The terms “therapeutically effective amount” and “effective amount” are meant to include the amount of a compound that, when administered, is sufficient to prevent development of, or alleviate to some extent, one or more of the symptoms of the disorder, disease, or condition being treated. The terms “therapeutically effective amount” or “effective amount” also refer to the amount of a compound that is sufficient to elicit the biological or medical response of a biological molecule (e.g., a protein, enzyme, RNA, or DNA), cell, tissue, system, animal, or human, which is being sought by a researcher, veterinarian, medical doctor, or clinician.

The terms “pharmaceutically acceptable carrier,” “pharmaceutically acceptable excipient,” “physiologically acceptable carrier,” and “physiologically acceptable excipient” refer to a pharmaceutically-acceptable material, composition, or vehicle, such as a liquid or solid filler, diluent, solvent, or encapsulating material. In one embodiment, each component is “pharmaceutically acceptable” in the sense of being compatible with other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition, Lippincott Williams & Wilkins: Philadelphia, PA, 2005; Handbook of Pharmaceutical Excipients, 5th Edition, Rowe et al., Eds., The Pharmaceutical Press and the American Pharmaceutical Association: 2005; and Handbook of Pharmaceutical Additives, 3rd Edition, Ash and Ash Eds., Gower Publishing Company: 2007; Pharmaceutical Preformulation and Formulation, 2nd Edition, Gibson Ed., CRC Press LLC: Boca Raton, FL, 2009.

The terms “about” and “approximately” mean an acceptable error for a particular value as determined by one of ordinary skill in the art, which depends in part on how the value is measured or determined. In certain embodiments, the terms “about” and “approximately” mean within 1, 2, 3, or 4 standard deviations. In certain embodiments, the terms “about” and “approximately” mean within 50%, 20%, 15%, 10%, 9%, 8%, 7%, 6%, 5%, 4%, 3%, 2%, 1%, 0.5%, or 0.05% of a given value or range.

The terms “active ingredient” and “active substance” refer to a compound, which is administered, alone or in combination with one or more pharmaceutically acceptable excipients, to a subject for treating, preventing, or ameliorating one or more symptoms of a disorder, disease, or condition. As used herein, “active ingredient” and “active substance” may be an optically active isomer of a compound described herein.

The terms “drug,” “therapeutic agent,” and “chemotherapeutic agent” refer to a compound, or a pharmaceutical composition thereof, which is administered to a subject for treating, preventing, or ameliorating one or more symptoms of a disorder, disease, or condition.

The terms “naturally occurring” and “native” when used in connection with biological materials such as nucleic acid molecules, polypeptides, host cells, and the like, refer to materials which are found in nature and are not manipulated by man. Similarly, “non-naturally occurring” or “non-native” refers to a material that is not found in nature or that has been structurally modified or synthesized by man.

The term “PI3K” refers to a phosphoinositide 3-kinase or variant thereof, which is capable of phosphorylating the inositol ring of PI in the D-3 position. The term “PI3K variant” is intended to include proteins substantially homologous to a native PI3K, i.e., proteins having one or more naturally or non-naturally occurring amino acid deletions, insertions, or substitutions (e.g., PI3K derivatives, homologs, and fragments), as compared to the amino acid sequence of a native PI3K. The amino acid sequence of a PI3K variant is at least about 80% identical, at least about 90% identical, or at least about 95% identical to a native PI3K. Examples of PI3K include, but are not limited to, p110α, p110β, p110δ, p110γ, PI3K-C2α, PI3K-C2β, PI3K-C2γ, Vps34, mTOR, ATM, ATR, and DNA-PK. See, Fry, Biochem. Biophys. Acta 1994, 1226, 237-268; Vanhaesebroeck and Waterfield, Exp. Cell. Res. 1999, 253, 239-254; and Fry, Breast Cancer Res. 2001, 3, 304-312. PI3Ks are classified into at least four classes. Class I includes p110α, p110β, p110δ, and p110γ. Class II includes PI3K-C2α, PI3K-C2β, and PI3K-C2γ. Class III includes Vps34. Class IV includes mTOR, ATM, ATR, and DNA-PK. In certain embodiments, the PI3K is a Class I kinase. In certain embodiments, the PI3K is p110α, p110β, p110δ, or p110γ. In certain embodiments, the PI3K is a variant of a Class I kinase. In certain embodiments, the PI3K is a p110α mutant. Examples of p110α mutants include, but are not limited to, R38H, G106V, K111N, K227E, N345K, C420R, P539R, E542K, E545A, E545G, E545K, Q546K, Q546P, E453Q, H710P, 1800L, T1025S, M10431, M1043V, H1047L, H1047R, and H1047Y (Ikenoue et al., Cancer Res. 2005, 65, 4562-4567; Gymnopoulos et al., Proc. Natl. Acad Sci., 2007, 104, 5569-5574). In certain embodiments, the PI3K is a Class II kinase. In certain embodiments, the PI3K is PI3K-C2α, PI3K-C2β, or PI3K-C2γ. In certain embodiments, the PI3K is a Class III kinase. In certain embodiments, the PI3K is Vps34. In certain embodiments, the PI3K is a Class IV kinase. In certain embodiments, the PI3K is mTOR, ATM, ATR, or DNA-PK.

The term “PD-1” refers to programmed cell death-1 receptor or programmed cell death protein 1, also known as cluster of differentiation 279 (CD279). The PD-1 protein in humans is encoded by the PDCD1 gene. PD-1 is a cell surface receptor that plays an important role in down-regulating the immune system and promoting self tolerance by suppressing T cell inflammatory activity. PD-1 is expressed on the surface of activated T cells and guards against autoimmunity through a dual mechanism of promoting apoptosis (programmed cell death) in antigen specific T-cells in lymph nodes while simultaneously reducing apoptosis in regulatory T cells (anti-inflammatory, suppressive T cells. PD-1 inhibitors activate the immune system to attack tumors and are therefore used to treat some types of cancer.

The term “PD-L1” refers to programed death-ligand 1 also known as cluster of differentiation 274 (CD274) or B7 homolog 1 (B7-H1). PD-L1 is a protein encoded by the CD274 gene. PD-L1 is a transmembrane protein that plays a major role in suppressing the immune system. PD-L1 binds to its receptor, PD-1 (found on activated T cells, B cells, and myeloid cells) to modulate activation or inhibition of T cell responses. PD-L1, the ligand for PD-1, is highly expressed in several cancers. Inhibition of the interaction between PD-1 and PD-L1 can enhance T-cell responses against cancer cells or tumors, and thus be useful for the treatment of cancer.

The terms “synergy,” “synergism,” and “synergistic” as used herein refer to a combination of therapies (e.g., use of a PI3K inhibitor of Compound I and a PD-1 inhibitor (e.g., pembrolizumab)) that is more effective than the expected additive effects of any two or more single therapies. For example, a synergistic effect of a combination of therapies permits the use of lower dosages of one or more of the therapies and/or less frequent administration of said therapies to a subject. The ability to utilize lower dosages of therapies and/or to administer the therapies less frequently reduces the toxicity associated with the administration of the therapies to a subject without reducing the efficacy of said therapies in the prevention, management, treatment, or amelioration of a given disease, such as an autoimmune disease, inflammatory disease, or cancer including, but not limited to, chronic lymphocytic leukemia or non-Hodgkin's lymphoma. In addition, a synergistic effect can result in improved efficacy of therapies in the prevention, management, treatment, or amelioration of a given disease, such an autoimmune disease, inflammatory disease, or cancer including, but not limited to, chronic lymphocytic leukemia or non-Hodgkin's lymphoma. Finally, synergistic effects of a combination of therapies may avoid or reduce adverse or unwanted side effects associated with the use of any single therapy. The “synergy,” “synergism,” or “synergistic” effect of a combination may be determined herein by the methods of Chou et al., and/or Clarke et al. See Ting-Chao Chou, Theoretical Basis, Experimental Design, and Computerized Simulation of Synergism and Antagonism in Drug Combination Studies, Pharmacol. Rev. 58:621-681 (2006), and Clarke et al., Issues in experimental design and endpoint analysis in the study of experimental cytotoxic agents in vivo in breast cancer and other models, Breast Cancer Research and Treatment 46:255-278 (1997), which are both incorporated by reference for the methods of determining the “synergy,” synergism,” or “synergistic” effect of a combination.

P3K inhibitor of Compound I

One therapeutic agent administered in the combination therapy provided herein is a PI3K inhibitor. In some embodiments, the PI3K inhibitor is Compound I or pharmaceutically acceptable salt or hydrate thereof, wherein Compound I has the structure of:

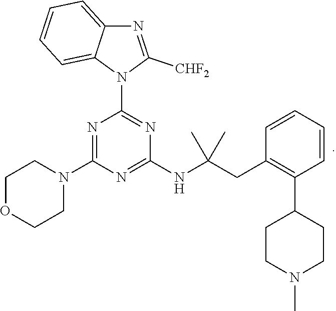 [SMILES: CN1CCC(c2ccccc2CC(C)(C)Nc2nc(N3CCOCC3)nc(-n3c(C(F)F)nc4ccccc43)n2)CC1]

### PD-1 Inhibitors

Another therapeutic agent administered in the combination therapy provided herein is a PD-1 inhibitor.

The PD-1 (programmed cell death-1) receptor (also known as CD279) is expressed on the surface of activated T cells. Its ligand, PD-L1, is commonly expressed on the surface of dendritic cells or macrophages. In some instances, PD1 and PD-L1 interaction halts or limits the development of the T cell response. When PD-L1 binds to PD-1, an inhibitory signal is transmitted into the T cell, which reduces cytokine production and suppresses T-cell proliferation. In some instances, cancer or tumor cells exploit this signaling pathway as a mechanism to evade detection and inhibit the immune response. In some instances, PD-L1 is overexpressed on cancer or tumor cells or on non-transformed cells in the tumor microenvironment. In some instances, PD-L1 expressed on the tumor cells binds to PD-1 receptors on the activated T cells, which leads to the inhibition of the cytotoxic T cells. These deactivated T cells remain inhibited in the tumor microenvironment. The PD1/PD-L1 pathway represents an adaptive immune resistance mechanism that is exerted by cancer or tumor cells in response to endogenous anti-tumor activity.

PD-1 inhibitors (or anti-PD-1 agents) and PD-L1 inhibitors (or anti-PD-L1 agents) block the interaction between PD-1 and PD-L1 and boost the immune response against cancer cells. In some instances, the blockade of receptor engagement results in the amplification of antigen-specific T cell responses against cancer cells. In some instances, antibodies that block the PD-1/PD-L1 interaction target lymphocyte receptors or their ligands in order to enhance endogenous antitumor activity. In some instances, PD-1 inhibitors and PD-L1 inhibitors overcome distinct immune suppressive pathways within the tumor microenvironment. In some instances, PD-1 inhibitors and/or PD-L1 inhibitors are useful for treating cancer.

Any suitable PD-1 inhibitor may be used in combination with a PI3K inhibitor described herein. In some embodiments, the PD-1 inhibitor is an antagonist of PD-1. In some embodiments, the PD-1 inhibitor is an antibody, variant, or biosimilar thereof. In some embodiments, the PD-1 inhibitor is a monoclonal antibody. In some embodiments, the method of treating cancer with a PI3K inhibitor described herein in combination with a PD-1 inhibitor provided herein results in a transient reduction in the level of systemic immunosuppression.

Some embodiments provided herein describe a pharmaceutical compositions or methods for use the pharmaceutical compositions comprising a PI3K inhibitor described herein in combination with a PD-1 inhibitor. PD-1 inhibitors for use in pharmaceutical compositions and methods provided herein include, but are not limited to, nivolumab (OPDIVO®), pembrolizumab (Keytruda®), MEDI0680 (AMP-514), AMP-224, AMP-514 (Amplimmune), BGB-A317, PDR001, REGN2810, JS001, AGEN2034, and variants and biosimilars thereof. In some embodiments, the PD-1 inhibitor is to nivolumab (Opdivo®), pembrolizumab (Keytruda®), MEDI0680 (AMP-514), AMP-224, AMP-514 (Amplimmune), or variants or biosimilars thereof. In some embodiments, the PD-1 inhibitor is pidilzumab (CT-011), or a variant or biosimilar thereof. In some embodiments, the PD-1 inhibitor is nivolumab (Opdivo®), or pembrolizumab (Keytruda®), or a variant or biosimilar thereof. In some embodiments, the PD-1 inhibitor is BGB-A317, a BGB-A317 variant, or a BGB-A317 biosimilar. In some embodiments, the PD-1 inhibitor is PDR001, a PDR001 variant, or a PDR001 biosimilar. In some embodiments, the PD-1 inhibitor is REGN2810, a REGN2810 variant, or a REGN2810 biosimilar.

In a preferred embodiment, the PD-1 inhibitor is pembrolizumab (Keytruda®), a pembrolizumab variant, or a pembrolizumab biosimilar.

In another embodiment, the PD-1 inhibitor is nivolumab (Opdivo®), a nivolumab variant, or a nivolumab biosimilar.

### Methods of Use

Provided herein are methods for treating a cancer in a patient in need thereof in one or more treatment cycles, comprising: (i) administering Compound I or a pharmaceutically acceptable salt thereof to the patient for a first period of time within each of the one or more treatment cycles; and (ii) administering a PD-1 inhibitor (e.g., pembrolizumab) to the patient after the administration of Compound I or a pharmaceutically acceptable salt thereof.

In some embodiments, Compound I or a pharmaceutically acceptable salt thereof and the PD-1 inhibitor (e.g., pembrolizumab) are sequentially administered in each of the one or more treatment cycles. In some embodiments of the sequential administration, Compound I or a pharmaceutically acceptable salt thereof is intermittently administered (e.g., there is an interval of at least 7, 8, 9, 10, 11, 12, 13, 14, 15, 16, 17, 18, 19, or 20 days between the days during which Compound I or a pharmaceutically acceptable salt thereof is administered). The sequential administration of Compound I or a pharmaceutically acceptable salt thereof and the PD-1 inhibitor (e.g., pembrolizumab) are more efficacious than concomitant administration of Compound I or a pharmaceutically acceptable salt thereof and the PD-1 inhibitor (e.g., pembrolizumab), e.g., Compound I or a pharmaceutically acceptable salt thereof is continuously administered and the PD-1 inhibitor (e.g., pembrolizumab) is also administered on one or more days on which Compound I or a pharmaceutically acceptable salt thereof is administered.

The term “intermittent” or “intermittently” as used herein is intended to mean stopping and starting at either regular or irregular intervals. For example, intermittent administration of the compound is administration for five days in every three-week treatment cycle, administration in treatment cycles (e.g., daily administration for five days in each treatment cycle of 3 weeks, then a rest period for 12 days with no administration).

In some embodiments, Compound I or a pharmaceutically acceptable salt thereof demonstrates higher avidity and biologic activity for a PI3K (e.g., PI3KS) compared to other PI3K inhibitors, including but not limited to idelalisib. In some embodiments, Compound I or pharmaceutically acceptable salts thereof demonstrates improved or superior drug distribution to blood cells compared to other PI3K inhibitors, including but not limited to idelalisib.

In some embodiments, the combination therapy of a PD-1 inhibitor (e.g., pembrolizumab) and Compound I or pharmaceutically acceptable salts thereof shows low toxicity to normal cells. In various embodiments, the combination of a PD-1 inhibitor (e.g., pembrolizumab) and Compound I or pharmaceutically acceptable salts thereof is selectively toxic or more toxic to rapidly proliferating cells, e.g., cancerous tumors, than to normal cells.

In some embodiments, the combination therapy described herein avoids or reduces adverse or unwanted, serious, or fatal side effects associated with the use of a PI3K inhibitor (e.g., idelalisib) and/or a PD-1. In some embodiments, the combination therapy described herein avoids, reduces, or minimizes (serious) infections, neutropenia, (severe) diarrhea, colon inflammation, colitis, lung tissue inflammation (pneumonitis), intestinal perforation, pneumonia, anemia, thrombocytopenia, nausea, fever, fatigue, cough, abdominal pain, chills, rash, vomiting, hypertriglyceridemia, hyperglycemia, elevated levels of liver enzymes (e.g., ALT and ALST), liver toxicity, swelling in extremities, or a combination thereof in patients receiving the combination therapy. In certain embodiments, the combination therapy described herein avoids, reduces, or minimizes the incidence of infection, including serious infection. In certain embodiments, the combination therapy described herein avoids, reduces, or minimizes the incidence of neutropenia. In certain embodiments, the combination therapy described herein avoids, reduces, or minimizes the incidence of diarrhea, including severe diarrhea. In certain embodiments, the combination therapy described herein avoids, reduces, or minimizes the incidence of colon inflammation. In certain embodiments, the combination therapy described herein avoids, reduces, or minimizes the incidence of colitis. In certain embodiments, the combination therapy described herein avoids, reduces, or minimizes the incidence of lung tissue inflammation (pneumonitis). In certain embodiments, the combination therapy described herein avoids, reduces, or minimizes the incidence of intestinal perforation. In certain embodiments, the combination therapy described herein avoids, reduces, or minimizes the incidence of pneumonia. In certain embodiments, the combination therapy described herein avoids, reduces, or minimizes the incidence of anemia. In certain embodiments, the combination therapy described herein avoids, reduces, or minimizes the incidence of thrombocytopenia. In certain embodiments, the combination therapy described herein avoids, reduces, or minimizes the incidence of nausea. In certain embodiments, the combination therapy described herein avoids, reduces, or minimizes the incidence of fever. In certain embodiments, the combination therapy described herein avoids, reduces, or minimizes the incidence of fatigue. In certain embodiments, the combination therapy described herein avoids, reduces, or minimizes the incidence of cough. In certain embodiments, the combination therapy described herein avoids, reduces, or minimizes the incidence of abdominal pain. In certain embodiments, the combination therapy described herein avoids, reduces, or minimizes the incidence of chills. In certain embodiments, the combination therapy described herein avoids, reduces, or minimizes the incidence of rash. In certain embodiments, the combination therapy described herein avoids, reduces, or minimizes the incidence of vomiting. In certain embodiments, the combination therapy described herein avoids, reduces, or minimizes the incidence of hypertriglyceridemia.

In certain embodiments, the combination therapy described herein avoids, reduces, or minimizes the incidence of hyperglycemia. In certain embodiments, the combination therapy described herein avoids, reduces, or minimizes the incidence of elevated levels of liver enzymes (e.g., ALT and ALST). In certain embodiments, the combination therapy described herein avoids, reduces, or minimizes the incidence of liver toxicity. In certain embodiments, the combination therapy described herein avoids, reduces, or minimizes the incidence of swelling in the extremities.

In some embodiments, the combination therapy described herein avoids or reduces adverse or unwanted side effects associated with chemotherapy, radiotherapy, or cancer therapy. In some instances, the combination therapies and/or compositions described herein provide chemo-protective and/or radio-protective properties to non-cancerous cells. In further or additional embodiments, the lower amount/doses of PI3K inhibitor reduces or minimizes any undesired side-effects associated with chemotherapy. Non-limiting examples of side-effects associated with chemotherapy, radiotherapy, or cancer therapy include fatigue, anemia, appetite changes, bleeding problems, diarrhea, constipation, hair loss, nausea, vomiting, pain, peripheral neuropathy, swelling, skin and nail changes, urinary and bladder changes, and trouble swallowing.

Some embodiments provided herein describe a method for treating or preventing a proliferative disease or disorder (e.g., a cancer) comprising administering a PI3K inhibitor in combination with a PD-1 inhibitor (e.g., pembrolizumab) inhibitor. In certain embodiments, provided herein are methods for treating or preventing a disease comprising administering a therapeutic effective amount of Compound I or a pharmaceutically acceptable salt thereof is administered and a therapeutic effective amount of the PD-1 inhibitor (e.g., pembrolizumab). In some embodiments, Compound I or a pharmaceutically acceptable salt thereof is administered at a dosage less than a therapeutic effective amount of administering Compound I or a pharmaceutically acceptable salt thereof alone. In some embodiments, the PD-1 inhibitor (e.g., pembrolizumab) is administered at a dosage less than a therapeutic effective amount of administering the PD-1 inhibitor (e.g., pembrolizumab) alone. In some embodiments, the co-administration of Compound I or pharmaceutically acceptable salt thereof and the PD-1 inhibitor (e.g., pembrolizumab) is effective in treating the cancer. In some embodiments, the PD-1 inhibitor is pembrolizumab or nivolumab. In one preferred embodiment, the PD-1 inhibitor is pembrolizumab.

In some embodiments, the proliferative disease or disorder is cancer. In certain embodiments, the proliferative disease or disorder is a hematological cancer. In certain embodiments, the proliferative disease or disorder is a solid tumor. In certain embodiments, the proliferative disease or disorder is a cancer of the breast, skin, prostate, cervix, uterus, ovary, testes, bladder, lung, liver, larynx, oral cavity, colon and gastrointestinal tract (e.g., esophagus, stomach, pancreas), brain, thyroid, blood, and lymphatic system.

In certain embodiments, the cancer treatable with the methods provided herein includes, but is not limited to, (1) leukemias, including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome or a symptom thereof (such as anemia, thrombocytopenia, neutropenia, bicytopenia or pancytopenia), refractory anemia (RA), RA with ringed sideroblasts (RARS), RA with excess blasts (RAEB), RAEB in transformation (RAEB-T), preleukemia, and chronic myelomonocytic leukemia (CMML), (2) chronic leukemias, including, but not limited to, chronic myelocytic (granulocytic) leukemia, chronic lymphocytic leukemia, and hairy cell leukemia; (3) polycythemia vera; (4) lymphomas, including, but not limited to, Hodgkin's disease and non-Hodgkin's disease; (5) multiple myelomas, including, but not limited to, smoldering multiple myeloma, non-secretory myeloma, osteosclerotic myeloma, plasma cell leukemia, solitary plasmacytoma, and extramedullary plasmacytoma; (6) Waldenstrom's macroglobulinernia; (7) monoclonal gammopathy of undetermined significance; (8) benign monoclonal gammopathy; (9) heavy chain disease; (10) bone and connective tissue sarcomas, including, but not limited to, bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, metastatic cancers, neurilemmoma, rhabdomyosarcoma, and synovial sarcoma; (11) brain tumors, including, but not limited to, glioma, astrocytoma, brain stem glioma, ependymoma, aligodendrogliorna, nonglial tumor, acoustic neurinoma, craniopharyngioma, medulloblastoma, meningioma, pineocytoma, pineoblastoma, and primary brain lymphoma; (12) breast cancer, including, but not limited to, adenocarcinoma, lobular (small cell) carcinoma, intraductal carcinoma, medullary breast cancer, mutinous breast cancer, tubular breast cancer, papillary breast cancer, primary cancers, Paget's disease, and inflammatory breast cancer; (13) adrenal cancer, including, but not limited to, pheochromocytom and adrenocortical carcinoma; (14) thyroid cancer, including, but not limited to, papillary or follicular thyroid cancer, medullary thyroid cancer, and anaplastic thyroid cancer; (15) pancreatic cancer, including, but not limited to, insulinoma, gastrinoma, glucagonoma, vipoma, somatostatin-secreting tumor, and carcinoid or islet cell tumor; (16) pituitary cancer, including, but limited to, Cushing's disease, prolactin-secreting tumor, acromegaly, and diabetes insipius; (17) eye cancer, including, but not limited, to ocular melanoma such as iris melanoma, choroidal melanoma, and ciliary body melanoma, and retinoblastoma; (18) vaginal cancer, including, but not limited to, squamous cell carcinoma, adenocarcinoma, and melanoma; (19) vulvar cancer, including, but not limited to, squamous cell carcinoma, melanoma, adenocarcinoma, basal cell carcinoma, sarcoma, and Paget's disease; (20) cervical cancers, including, but not limited to, squamous cell carcinoma, and adenocarcinoma; (21) uterine cancer, including, but not limited to, endometrial carcinoma and uterine sarcoma; (22) ovarian cancer, including, but not limited to, ovarian epithelial carcinoma, borderline tumor, germ cell tumor, and stromal tumor; (23) esophageal cancer, including, but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; (24) stomach cancer, including, but not limited to, adenocarcinoma, fungating (polypoid), ulcerating, superficial spreading, diffusely spreading, malignant lymphoma, liposarcoma, fibrosarcoma, and carcinosarcoma; (25) colon cancer; (26) rectal cancer; (27) liver cancer, including, but not limited to, hepatocellular carcinoma and hepatoblastoma; (28) gallbladder cancer, including, but not limited to, adenocarcinoma; (29) cholangiocarcinoma, including, but not limited to, papillary, nodular, and diffuse; (30) lung cancer, including, but not limited to, non-small cell lung cancer, squamous cell carcinoma (epidermoid carcinoma), adenocarcinoma, large-cell carcinoma, and small-cell lung cancer; (31) testicular cancer, including, but not limited to, germinal tumor, seminoma, anaplastic, classic (typical), spermatocytic, nonserninoma, embryonal carcinoma, teratoma carcinoma, and choriocarcinoma (yolk-sac tumor); (32) prostate cancer, including, but not limited to, adenocarcinoma, leiomyosarcoma, and rhabdomyosarcorna; (33) penal cancer; (34) oral cancer, including, but not limited to, squamous cell carcinoma; (35) basal cancer; (36) salivary gland cancer, including, but not limited to, adenocarcinoma, mucoepidermoid carcinoma, and adenoidcystic carcinoma; (37) pharynx cancer, including, but not limited to, squamous cell cancer and verrucous; (38) skin cancer, including, but not limited to, basal cell carcinoma, squamous cell carcinoma and melanoma, superficial spreading melanoma, nodular melanoma, lentigo malignant melanoma, and acral lentiginous melanoma; (39) kidney cancer, including, but not limited to, renal cell cancer, adenocarcinoma, hypernephroma, fibrosarcoma, and transitional cell cancer (renal pelvis and/or uterer); (40) Wilms' tumor; (41) bladder cancer, including, but not limited to, transitional cell carcinoma, squamous cell cancer, adenocarcinoma, and carcinosarcoma; (42) reproductive cancers, such as cervical cancer, uterus cancer, ovarian cancer, or testicular cancer; (43) esophagus cancer; (44) laryngeal cancer; (45) head and neck cancer (such as mouth, nose, throat, larynx, sinuses, or salivary glands cancer); and other cancer, including, not limited to, myxosarcoma, osteogenic sarcoma, endotheliosarcoma, lymphangio-endotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma, and papillary adenocarcinomas (See Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America). In some embodiments, the cancer is non-small cell lung cancer, melanoma, renal cell cancer, head and neck cancer, colon cancer, or mesothelioma. In some embodiments, the cancer is non-small cell lung cancer. In some embodiments, the cancer is melanoma.

In certain embodiments, provided herein are methods of treating a hematological malignancy with a combination of an effective amount of Compound I or a pharmaceutically acceptable salt thereof and an effective amount of PD-1 inhibitor in a patient. In certain embodiments, the hematological malignancy is a leukemia, a lymphoma, a myeloma, a non-Hodgkin's lymphoma, a Hodgkin's lymphoma, T-cell malignancy, or a B-cell malignancy. In some embodiments, the hematological malignancy is Hodgkin's lymphoma. In some embodiments, the hematological malignancy is chronic lymphocytic leukemia, follicular lymphoma, diffuse large B-cell lymphoma, or non-Hodgkin's lymphoma. In some embodiments, the hematological malignancy is chronic lymphocytic leukemia or non-Hodgkin's lymphoma. In some embodiments, the hematological malignancy is chronic lymphocytic leukemia. In other embodiments, the hematological malignancy is non-Hodgkin's lymphoma. In some embodiments, the hematological malignancy is follicular lymphoma. In other embodiments, the hematological malignancy is diffuse large B-cell lymphoma.

In certain embodiments, the hematological malignancy is a T-cell malignancy. In certain embodiments, T-cell malignancies include peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

In certain embodiments, the hematological malignancy is a B-cell malignancy. In certain embodiments, B-cell malignancies include acute lymphoblastic leukemia (ALL), acute myelogenous leukemia (AML), chronic myelogenous leukemia (CML), acute monocytic leukemia (AMoL), chronic lymphocytic leukemia (CLL), high-risk chronic lymphocytic leukemia (CLL), small lymphocytic lymphoma (SLL), high-risk small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In certain embodiments, the B-cell malignancy is diffuse large B-cell lymphoma (DLBCL). In certain embodiments, the hematological malignancy is diffuse large B-cell lymphoma (DLBCL). In certain embodiments, the DLBCL is an activated B-cell DLBCL (ABC-DLBCL), a germinal center B-cell like DLBCL (GBC-DLBCL), a double hit DLBCL (DH-DLBCL), or a triple hit DLBCL (TH-DLBCL). In certain embodiments, the hematological malignancy is relapsed-refractory diffuse large B-cell lymphoma (r/r DLBCL).

In certain embodiments, the hematological malignancy is a relapsed or refractory hematological malignancy. In certain embodiments, the relapsed or refractory hematological malignancy is a relapsed or refractory T-cell malignancy. In certain embodiments, the relapsed or refractory hematological malignancy is a relapsed or refractory B-cell malignancy.

In other embodiments, the disease or disorder is a solid tumor cancer. In some embodiments, the solid tumor cancer is selected from the group consisting of a carcinoma, an adenocarcinoma, an adrenocortical carcinoma, a colon adenocarcinoma, a colorectal adenocarcinoma, a colorectal carcinoma, a ductal cell carcinoma, a lung carcinoma, a thyroid carcinoma, a nasopharyngeal carcinoma, a melanoma, a non-melanoma skin carcinoma, and a lung cancer.

In certain embodiments, the cancer is lung cancer (e.g., non-small cell lung cancer), melanoma, renal cell cancer, head and neck cancer, breast cancer (e.g., triple-negative breast cancer), non-Hodgkin's lymphoma, Hodgkin lymphoma, colorectal cancer, urothelial carcinoma, gastric cancer, cervical cancer (e.g., advanced cervical cancer), hepatocellular carcinoma, Merkel cell carcinoma (e.g., advanced Merkel cell carcinoma), esophageal cancer, endometrial cancer, cutaneous squamous cell carcinoma, or mesothelioma.

In one embodiment, the cancer is melanoma. In another embodiment, the cancer is lung cancer. In one specific embodiment, the lung cancer is non-small cell lung cancer. In another embodiment, the cancer is renal cell cancer. In yet another embodiment, the cancer hepatocellular carcinoma. In yet another embodiment, the cancer is colorectal cancer. In another specific embodiment, the colorectal cancer is microsatellite instability-high or mismatch repair deficient colorectal cancer. In yet another embodiment, the cancer is urothelial carcinoma (e.g. advanced Urothelial Carcinoma). In yet another embodiment, the cancer is Advanced Endometrial Carcinoma. In yet another embodiment, the cancer is a tumor mutational burden-high cancer.

In some embodiments of the methods provided herein, the methods further comprise (iii) evaluating efficacy of the administration of the combination of Compound I or a pharmaceutically acceptable salt thereof and the PD-1 inhibitor by measuring levels of regulatory T-cells (CD25hi FoxP3+ T cells) or effector T cells (e.g., CD8+ and CD4+ cells). In some embodiments, the when the regulatory T-cells (CD25hi FoxP3+ T cells) or effector T cells (e.g., CD8+ and CD4+ cells) is suppressed to be lower than a certain threshold (e.g., by at least 35%) in comparison to the baseline level, it is indicative of efficacious treatment (e.g., combination treatment). In some embodiments, a level of regulatory T-cells (CD25hi FoxP3+ T cells) or effector T cells (e.g., CD8+ and CD4+ cells) that is less than or equal to about 70%, 65%, 60%, 55%, 50%, 45%, 40%, 35%, 30%, 25%, 20%, 15%, or 10% of a baseline level of the regulatory T-cells (CD25hi FoxP3+ T cells) or effector T cells (e.g., CD8+ and CD4+ cells) indicates that the one or more treatment cycles of administration of the administration of the combination of Compound I or a pharmaceutically acceptable salt thereof and the PD-1 inhibitor is effective. In some embodiments, a level of regulatory T-cells (CD25hi FoxP3+ T cells) or effector T cells (e.g., CD8+ and CD4+ cells) that is less than or equal to about 65% of a baseline level of the regulatory T-cells (CD25hi FoxP3+ T cells) or effector T cells (e.g., CD8+ and CD4+ cells) indicates that the one or more treatment cycles of administration of the administration of the combination of Compound I or a pharmaceutically acceptable salt thereof and the PD-1 inhibitor is effective.

Depending on the disorder, disease, or condition to be treated, and the patient's condition, Compound I or pharmaceutically acceptable salt or the PD-1 inhibitor (e.g., pembrolizumab) can be administered by oral, parenteral (e.g., intramuscular, intraperitoneal, intravenous, ICV, intracistemal injection or infusion, subcutaneous injection, or implant), inhalation, nasal, vaginal, rectal, sublingual, or topical (e.g., transdermal or local) routes of administration and can be formulated, alone or together, in suitable dosage unit with pharmaceutically acceptable excipients, carriers, adjuvants, and vehicles appropriate for each route of administration as described elsewhere herein.

### Dosages and Dosing Regimens

In certain embodiments, the methods provided herein comprise administering Compound I or a pharmaceutically acceptable salt thereof and a PD-1 inhibitor (e.g., pembrolizumab) to the patient simultaneously or sequentially. In preferred embodiments, Compound I or a pharmaceutically acceptable salt thereof and the PD-1 inhibitor (e.g., pembrolizumab) are administered sequentially.

The suitability of a particular route of administration employed for a particular active agent will depend on the active agent itself (e.g., whether it can be administered orally without decomposing prior to entering the blood stream) and the disease being treated. Recommended routes of administration for the second active agents are known to those of ordinary skill in the art. See, e.g., Physicians' Desk Reference, 1755-1760 (56th ed., 2002).

In certain embodiments, Compound I or a pharmaceutically acceptable salt thereof and a PD-1 inhibitor (e.g., pembrolizumab) are administered simultaneously, at essentially the same time, or sequentially. If administration takes place sequentially, the PD-1 inhibitor may be administered before or after administration of Compound I or a pharmaceutically acceptable salt thereof. In some preferred embodiments, the PD-1 inhibitor is administered after administration of Compound I or a pharmaceutically acceptable salt thereof. In some embodiments, the PD-1 inhibitor is administered simultaneously with administration of Compound I or a pharmaceutically acceptable salt thereof. In some embodiments, the PD-1 inhibitor is administered before the administration of Compound I or a pharmaceutically acceptable salt thereof.

In certain embodiments, Compound I or a pharmaceutically acceptable salt thereof and the PD-1 inhibitor (e.g., pembrolizumab) need not be administered by means of the same vehicle. In some embodiments, Compound I or a pharmaceutically acceptable salt thereof and the PD-1 inhibitor (e.g., pembrolizumab) are administered in different vehicles. The PD-1 inhibitor (e.g., pembrolizumab) may be administered one or more times, and the number of administrations of each component of the combination may be the same or different. In addition, Compound I or a pharmaceutically acceptable salt thereof and the PD-1 inhibitor (e.g., pembrolizumab) need not be administered at the same site.

In another aspect, provided herein are methods for treating a cancer in a patient in need thereof in one or more treatment cycles, comprising: (i) administering Compound I or a pharmaceutically acceptable salt thereof to the patient on Days 1 to 5 of each of the one or more treatment cycles; and (ii) administering a PD-1 inhibitor to the patient on Day 22 of each of the treatment cycles; wherein each of the one or more treatment cycles is 6 weeks; and wherein Compound I has the structure of:

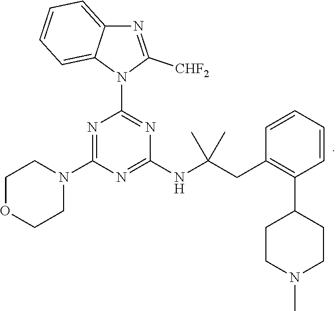 [SMILES: CN1CCC(c2ccccc2CC(C)(C)Nc2nc(N3CCOCC3)nc(-n3c(C(F)F)nc4ccccc43)n2)CC1]

In another aspect, provided herein are methods for treating a cancer in a patient in need thereof in one or more treatment cycles, comprising: (i) administering Compound I or a pharmaceutically acceptable salt thereof to the patient on Days 1 to 5 of each of the one or more treatment cycles; and (ii) administering a PD-1 inhibitor to the patient on Day 14 of each of the treatment cycles; wherein each of the one or more treatment cycles is 3 weeks; and wherein Compound I has the structure of:

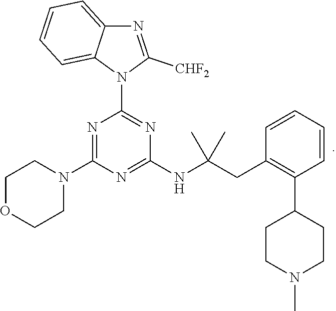 [SMILES: CN1CCC(c2ccccc2CC(C)(C)Nc2nc(N3CCOCC3)nc(-n3c(C(F)F)nc4ccccc43)n2)CC1]

### Treatment Cycles

In some preferred embodiments, the methods described herein further comprise administering Compound I or a pharmaceutically acceptable salt thereof and the PD-1 inhibitor (e.g., pembrolizumab) to the patient in need thereof in one or more treatment cycles repeated on a regular schedule optionally with a period of rest in between two treatment cycles. For example, in some instances, the combination treatment provided herein is given for 3 weeks followed by no period rest prior to the next treatment cycle. In some instances, the combination treatment provided herein is given for 6 weeks followed by no period rest prior to the next treatment cycle. In other embodiments, the combination treatment provided herein is given for 3 weeks followed by 1 week of rest prior to the next treatment cycle. In other embodiment, the combination treatment provided herein is given for 6 weeks followed by 3 week of rest prior to the next treatment cycle.

In some instances, a cycle comprises administration of Compound I or a pharmaceutically acceptable salt thereof at the same time as administration of the PD-1 inhibitor (e.g., pembrolizumab). In some instances, a treatment cycle comprises administration of Compound I or a pharmaceutically acceptable salt thereof on different days than the day(s) on which the PD-1 inhibitor (e.g., pembrolizumab) is administered. In some instances, Compound I or a pharmaceutically acceptable salt thereof and the PD-1 inhibitor (e.g., pembrolizumab) are administered for about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, or about 28 days apart.

In some instances, a treatment cycle comprises administration of Compound I or a pharmaceutically acceptable salt thereof for a first period of time followed by a second period of time. In some embodiment, which administration of the PD-1 inhibitor occurs on the next day of the second period of time. In some instances, the first period of time is about 1 day, about 2 days, about 3 days, about 4 days, about 5 days, about 6 days, about 7 days, about 8 days, about 9 days, about 10 days, about 11 days, about 12 days, about 13 days, or about 14 days. In some embodiment, the first period of time is 3 days, 4 days, 5 days, 6 days, or 1 week. In one preferred embodiment, the first period of time is 5 days.

In some embodiments, the first period starts on Day 1, Day 2, Day 3, Day 4, Day 5, Day 6, Day 7, or Day 8 of each of the one or more treatment cycles.

In some embodiment, the second period of time is 0 day, about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 3 weeks, about 4 weeks, about 5 weeks, about 6 weeks, about 7 weeks, or about 8 weeks. In one preferred embodiment, the second period of time is 16 days. In another embodiment, the second period of time is about 3 weeks.

In some embodiment, the PD-1 inhibitor is administered starting on the next day after the second period of time, wherein the second period of time is immediately after the first period.

In some embodiment, wherein the PD-1 inhibitor is administered about 7, 8, 9, 10, 11, 12, 13, 14, 15, 16, 17, 18, 19, or 20 days after the first period of time. In one preferred embodiment, wherein the PD-1 inhibitor is administered about 8 days after the first period of time. In another preferred embodiment, wherein the PD-1 inhibitor is administered about 16 days after the first period of time.

In some embodiments, the PD-1 inhibitor is administered for at least 6 weeks. In some embodiments, the PD-1 inhibitor is administered for 6, 9, 12, 15, 18, 21, 24, 27, or 30 weeks. In some embodiments, the PD-1 inhibitor is administered once every 3 weeks or once every 6 weeks. In some embodiments, the PD-1 inhibitor is administered at a dosage of about 200 mg or about 400 mg. In one embodiment, the PD-1 inhibitor is administered at a dosage of about 200 mg. In one embodiment, the PD-1 inhibitor is administered at a dosage of about 400 mg.

In some embodiments, the PD-1 inhibitor is administered intravenously (e.g., infusion).

In some embodiments, Compound I or a pharmaceutically acceptable salt thereof is administered at about 60 mg/day for 5 days in combination with pembrolizumab at the dose of about 400 mg once of each 6-week treatment cycle. In some specific embodiments, Compound I or a pharmaceutically acceptable salt thereof is administered at about 60 mg/day on Days 1-5 in combination with pembrolizumab at the dose of about 400 mg on Day 22 of each 6-week treatment cycle. In some embodiments, Compound I or a pharmaceutically acceptable salt thereof and pembrolizumab are administered for 1 to 10 treatment cycles. In some embodiments, Compound I or a pharmaceutically acceptable salt thereof and pembrolizumab are administered for 1 treatment cycle. In some embodiments, Compound I or a pharmaceutically acceptable salt thereof and pembrolizumab are administered for 2 treatment cycles. In some embodiments, Compound I or a pharmaceutically acceptable salt thereof and pembrolizumab are administered for 3 treatment cycles. In some embodiments, Compound I or a pharmaceutically acceptable salt thereof and pembrolizumab are administered for 4 treatment cycles. In some embodiments, Compound I or a pharmaceutically acceptable salt thereof and pembrolizumab are administered for 5 treatment cycles. In some embodiments, Compound I or a pharmaceutically acceptable salt thereof and pembrolizumab are administered for 6 treatment cycles. In some embodiments, Compound I or a pharmaceutically acceptable salt thereof and pembrolizumab are administered for 7 treatment cycles. In some embodiments, Compound I or a pharmaceutically acceptable salt thereof and pembrolizumab are administered for 8 treatment cycles. In some embodiments, Compound I or a pharmaceutically acceptable salt thereof and pembrolizumab are administered for 9 treatment cycles. In some embodiments, Compound I or a pharmaceutically acceptable salt thereof and pembrolizumab are administered for 10 treatment cycles. In some embodiments, Compound I or a pharmaceutically acceptable salt thereof and pembrolizumab are administered for more than 10 treatment cycles.

In some embodiments, Compound I or a pharmaceutically acceptable salt thereof is administered at about 60 mg/day for 5 days in combination with pembrolizumab at the dose of about 200 mg once of each 3-week treatment cycle. In some specific embodiments, Compound I or a pharmaceutically acceptable salt thereof is administered at about 60 mg/day on Days 1-5 in combination with pembrolizumab at the dose of about 200 mg on Day 14 of each 3-week treatment cycle. In some embodiments, Compound I or a pharmaceutically acceptable salt thereof and pembrolizumab are administered for 1 to 10 treatment cycles. In some embodiments, Compound I or a pharmaceutically acceptable salt thereof and pembrolizumab are administered for 1 treatment cycle. In some embodiments, Compound I or a pharmaceutically acceptable salt thereof and pembrolizumab are administered for 2 treatment cycles. In some embodiments, Compound I or a pharmaceutically acceptable salt thereof and pembrolizumab are administered for 3 treatment cycles. In some embodiments, Compound I or a pharmaceutically acceptable salt thereof and pembrolizumab are administered for 4 treatment cycles. In some embodiments, Compound I or a pharmaceutically acceptable salt thereof and pembrolizumab are administered for 5 treatment cycles. In some embodiments, Compound I or a pharmaceutically acceptable salt thereof and pembrolizumab are administered for 6 treatment cycles. In some embodiments, Compound I or a pharmaceutically acceptable salt thereof and pembrolizumab are administered for 7 treatment cycles. In some embodiments, Compound I or a pharmaceutically acceptable salt thereof and pembrolizumab are administered for 8 treatment cycles. In some embodiments, Compound I or a pharmaceutically acceptable salt thereof and pembrolizumab are administered for 9 treatment cycles. In some embodiments, Compound I or a pharmaceutically acceptable salt thereof and pembrolizumab are administered for 10 treatment cycles. In some embodiments, Compound I or a pharmaceutically acceptable salt thereof and pembrolizumab are administered for more than 10 treatment cycles.

In some embodiments, Compound I or a pharmaceutically acceptable salt thereof is administered at about 60 mg/day for 10 days in combination with pembrolizumab at the dose of about 200 mg twice of each 6-week treatment cycle. In some specific embodiments, Compound I or a pharmaceutically acceptable salt thereof is administered at about 60 mg/day on Days 1-5 and Days 22-26 in combination with pembrolizumab at the dose of about 200 mg on Day 14 and Day 35 of each 6-week treatment cycle. In some embodiments, Compound I or a pharmaceutically acceptable salt thereof and pembrolizumab are administered for 1 to 10 treatment cycles. In some embodiments, Compound I or a pharmaceutically acceptable salt thereof and pembrolizumab are administered for 1 treatment cycle. In some embodiments, Compound I or a pharmaceutically acceptable salt thereof and pembrolizumab are administered for 2 treatment cycles. In some embodiments, Compound I or a pharmaceutically acceptable salt thereof and pembrolizumab are administered for 3 treatment cycles. In some embodiments, Compound I or a pharmaceutically acceptable salt thereof and pembrolizumab are administered for 4 treatment cycles. In some embodiments, Compound I or a pharmaceutically acceptable salt thereof and pembrolizumab are administered for 5 treatment cycles. In some embodiments, Compound I or a pharmaceutically acceptable salt thereof and pembrolizumab are administered for 6 treatment cycles. In some embodiments, Compound I or a pharmaceutically acceptable salt thereof and pembrolizumab are administered for 7 treatment cycles. In some embodiments, Compound I or a pharmaceutically acceptable salt thereof and pembrolizumab are administered for 8 treatment cycles. In some embodiments, Compound I or a pharmaceutically acceptable salt thereof and pembrolizumab are administered for 9 treatment cycles. In some embodiments, Compound I or a pharmaceutically acceptable salt thereof and pembrolizumab are administered for 10 treatment cycles. In some embodiments, Compound I or a pharmaceutically acceptable salt thereof and pembrolizumab are administered for more than 10 treatment cycles.

In some instances, a cycle comprises administration of Compound I or a pharmaceutically acceptable salt thereof only. In some instances, Compound I or a pharmaceutically acceptable salt thereof is administered for about 1 day, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, or about 28 days. In one preferred embodiment, Compound I or a pharmaceutically acceptable salt thereof is administered for about 5 days. In another preferred embodiment, Compound I or a pharmaceutically acceptable salt thereof is administered for about 5 consecutive days.

In some embodiments, each of the one or more treatment cycles is 6 weeks, Compound I or a pharmaceutically acceptable salt thereof is administered on Days 1 to 5, and then the PD-1 inhibitor is administered once on Day 22.

In some embodiments, each of the one or more treatment cycles is 3 weeks, Compound I or a pharmaceutically acceptable salt thereof is administered on Days 1 to 5, and then the PD-1 inhibitor is administered once on Day 14.

In some instances, a treatment cycle comprises administration of the PD-1 inhibitor only. In some instances, the PD-1 inhibitor is administered for 1, 2, 3, or 4 times in the treatment cycle. In one preferred embodiment, the PD-1 inhibitor is administered once in the treatment cycle.

In some instances, the method for multiple treatment cycles the administration of a second treatment cycle within about 60 days or about 3 months. In some instances, the method for multiple treatment cycles comprises the administration of a second cycle within 50 days of the previous treatment cycle. In another instance, the second treatment cycle is administered within 45, 40, 35, 30, 25, 21, 20, 15, 14, 10, 9, 8, 7, 6, 5, 4, 3, 2 or 1 day(s) of the first treatment cycle. In some embodiments, the administration of any additional treatment cycles is within 10 days of the previous treatment cycle. In some embodiments, the administration of any additional treatment cycles is within 9 days of the previous treatment cycle. In some embodiments, the administration of any additional treatment cycles is about 3 weeks of the previous treatment cycle. In some embodiments, the administration of any additional treatment cycles is within 7 days of the previous treatment cycle. In some embodiments, the administration of any additional treatment cycles is within 6 days of the previous treatment cycle. In some embodiments, the administration of any additional treatment cycles is within 5 days of the previous treatment cycle. In some embodiments, the administration of any additional cycles is within 4 days of the previous treatment cycle. In some embodiments, the administration of any additional cycles is within 3 days of the previous treatment cycle. In some embodiments, the administration of any additional treatment cycles is within 2 days of the previous treatment cycle. In some embodiments, the administration of any additional treatment cycles is within 1 day of the previous treatment cycle.

The length of a treatment cycle depends on the treatment being given. In some embodiments, the length of a treatment cycle ranges from two to six weeks. In some embodiments, the length of a treatment cycle ranges from four to six weeks. In some embodiments, the length of a treatment cycle is 3 weeks. In some embodiments, the length of a treatment cycle is 6 weeks. In some embodiments, a treatment cycle lasts one, two, three, or four weeks. In some embodiments, a treatment cycle lasts four weeks. The number of treatment doses scheduled within each cycle also varies depending on the drugs being given.

In some instances, the method of multiple treatment cycles compounds comprises administering Compound I or a pharmaceutically acceptable salt thereof and the PD-1 inhibitor in alternative treatment cycles. In some embodiment, Compound I or a pharmaceutically acceptable salt thereof is administered in treatment Cycles 1, 3, 5, 7, 9, and 11, and the PD-1 inhibitor is administered in treatment Cycles 2, 4, 6, 8, 10, and 12.

In some instances, the method for the administration of multiple compounds occurs in a sequential order, wherein Compound I or a pharmaceutically acceptable salt thereof is administered before the PD-1 inhibitor (e.g., pembrolizumab). In another instance, the PD-1 inhibitor (e.g., pembrolizumab) is administered before Compound I or a pharmaceutically acceptable salt thereof.

In some instances, the method for administering Compound I or a pharmaceutically acceptable salt thereof is oral and the method for administering the PD-1 inhibitor (e.g., pembrolizumab) is by injection. In some instances, the method for administering Compound I or a pharmaceutically acceptable salt thereof is by inhalation and the method for administering the PD-1 inhibitor (e.g., pembrolizumab) is by injection. In some instances, the method for administering Compound I or a pharmaceutically acceptable salt thereof is by injection and the method for administering the PD-1 inhibitor (e.g., pembrolizumab) is by injection.

In some embodiments, Compound I or a pharmaceutically acceptable salt thereof is administered daily, every other day, every other day 3 times a week, every 2 weeks, every 3 weeks, every 4 weeks, every 5 weeks, every 3 days, every 4 days, every 5 days, every 6 days, weekly, bi-weekly, 3 times a week, 4 times a week, 5 times a week, 6 times a week, once a month, twice a month, 3 times a month, once every 2 months, once every 3 months, once every 4 months, once every 5 months, or once every 6 months. In some preferred embodiments, Compound I or a pharmaceutically acceptable salt thereof is administered daily.

In some embodiments, the PD-1 inhibitor is administered daily, every other day, every 3 days, every 4 days, every 5 days, every 6 days, weekly, every 2 weeks, every 3 weeks, every 4 weeks, every 5 weeks, bi-weekly, 3 times a week, 4 times a week, 5 times a week, 6 times a week, once a month, twice a month, 3 times a month, once every 2 months, once every 3 months, once every 4 months, once every 5 months, or once every 6 months. In some preferred embodiments, the PD-1 inhibitor is administered once every 3 weeks (Q3W) or once every 6 weeks (Q6W).

In some embodiments, Compound I or a pharmaceutically acceptable salt thereof or the PD-1 inhibitor is optionally given continuously; alternatively, the dose of the administered therapeutic agent (Compound I or a pharmaceutically acceptable salt thereof or the PD-1 inhibitor) being administered is temporarily reduced or temporarily suspended for a certain length of time (i.e., a “drug holiday”). In some embodiments, the length of the drug holiday varies between 2 days and 1 year, including by way of example only, 2 days, 3 days, 4 days, 5 days, 6 days, 7 days, 8 days, 9 days, 10 days, 12 days, 14 days, 15 days, 20 days, 21 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

In certain embodiments, in the treatment, prevention, or amelioration of one or more symptoms of the disorders, diseases, or conditions described herein, an appropriate dosage level of Compound I or a pharmaceutically acceptable salt thereof generally is ranging from about 1 to about 1000 mg, from about 1 to about 500 mg, from about 5 to about 500 mg, from about 5 to about 200 mg, from about 5 to about 250 mg or from about 10 to about 150 mg which can be administered in single or multiple doses. In certain embodiments, Compound I or a pharmaceutically acceptable salt thereof is administered in an amount of about 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 225, 250, 275, 300, 325, 350, 375, 400, 450 or 500 mg. In certain embodiments, Compound I or a pharmaceutically acceptable salt thereof in an amount of about 1, about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 225, about 250, about 275, about 300, about 325, about 350, about 375, about 400, about 450, or about 500 mg/day. In certain embodiments, Compound I or a pharmaceutically acceptable salt thereof is administered in an amount of about 30 mg/day. In certain embodiments, Compound I or a pharmaceutically acceptable salt thereof is administered in an amount of about 45 mg/day. In certain embodiments, Compound I or a pharmaceutically acceptable salt thereof is administered in an amount of about 60 mg/day. In certain embodiments, Compound I or a pharmaceutically acceptable salt thereof is administered in an amount of about 90 mg/day. In certain embodiments, Compound I or a pharmaceutically acceptable salt thereof is administered in an amount of about 120 mg/day. In certain embodiments, Compound I or a pharmaceutically acceptable salt thereof is administered in an amount of about 150 mg/day. In certain embodiments, Compound I or a pharmaceutically acceptable salt thereof is administered in an amount of about 180 mg/day. In some preferred embodiments, Compound I or a pharmaceutically acceptable salt thereof is administered in an amount of about 60 mg/day.

For oral administration, the pharmaceutical compositions provided herein can be formulated in the form of tablets or capsules containing from about 1.0 to about 1,000 mg of Compound I or a pharmaceutically acceptable salt thereof, in one embodiment, about 1, about 5, about 10, about 15, about 20, about 25, about 50, about 75, about 100, about 150, about 200, about 250, about 300, about 400, about 500, about 600, about 750, about 800, about 900, and about 1,000 mg of Compound I or a pharmaceutically acceptable salt thereof for the symptomatic adjustment of the dosage to the patient to be treated. In some preferred embodiments, the pharmaceutical compositions provided herein can be formulated in the form of tablets containing about 60 mg of Compound I or a pharmaceutically acceptable salt thereof. The pharmaceutical compositions comprising Compound I or a pharmaceutically acceptable salt thereof is administered on a regimen of 1 to 4 times per day, including once, twice, three times, and four times per day. In some preferred embodiments, Compound I or a pharmaceutically acceptable salt thereof is administered once per day. In some embodiments, about 30 mg, about 45 mg, or about 60 mg of Compound I or a pharmaceutically acceptable salt thereof is administered once per day. In some embodiments, Compound I or a pharmaceutically acceptable salt thereof is administered to a patient in need thereof in an amount of about 60 mg daily for 1, 2, 3, 4, 5, 6, 7 consecutive days. In some embodiments, Compound I or a pharmaceutically acceptable salt thereof is administered to a patient in need thereof in an amount of about 60 mg daily for 5 consecutive days. In some embodiments, Compound I or a pharmaceutically acceptable salt thereof is administered to a patient in need thereof in an amount of about 60 mg daily for 2 consecutive days in a treatment cycle. In some embodiments, Compound I or a pharmaceutically acceptable salt thereof is administered to a patient in need thereof in an amount of about 60 mg daily for 3 consecutive days in a treatment cycle. In some embodiments, Compound I or a pharmaceutically acceptable salt thereof is administered to a patient in need thereof in an amount of about 60 mg daily for 4 consecutive days in a treatment cycle. In some embodiments, Compound I or a pharmaceutically acceptable salt thereof is administered to a patient in need thereof in an amount of about 60 mg daily for 6 consecutive days in a treatment cycle. In some embodiments, Compound I or a pharmaceutically acceptable salt thereof is administered to a patient in need thereof in an amount of about 60 mg daily for 7 consecutive days in a treatment cycle.

In some embodiments, Compound I or a pharmaceutically acceptable salt thereof is administered in Week 1 of a treatment cycle. In some embodiments, Compound I or a pharmaceutically acceptable salt thereof is administered in Week 2 of a treatment cycle. In some embodiments, Compound I or a pharmaceutically acceptable salt thereof is administered in Week 3 of a treatment cycle. In some embodiments, Compound I or a pharmaceutically acceptable salt thereof is administered in Week 4 of a treatment cycle. In some embodiments, Compound I or a pharmaceutically acceptable salt thereof is administered in Week 5 of a treatment cycle. In some embodiments, Compound I or a pharmaceutically acceptable salt thereof is administered in Week 6 of a treatment cycle. In some embodiments, Compound I or a pharmaceutically acceptable salt thereof is administered in Week 7 of a treatment cycle. In some embodiments, Compound I or a pharmaceutically acceptable salt thereof is administered in Week 8 of a treatment cycle. In some embodiments, Compound I or a pharmaceutically acceptable salt thereof is administered in Week 9 of a treatment cycle. In some embodiments, the treatment cycle is 3 weeks, 4 weeks, 5 weeks, 6 weeks, 7 weeks, 8 weeks, or 9 weeks.

In some embodiment, Compound I or a pharmaceutically acceptable salt thereof is administered in Week 1 of a treatment cycle of 3 weeks. In some embodiment, Compound I or a pharmaceutically acceptable salt thereof is administered in Week 2 of a treatment cycle of 3 weeks. In some embodiment, Compound I or a pharmaceutically acceptable salt thereof is administered in Week 3 of a treatment cycle of 3 weeks.

In some embodiment, Compound I or a pharmaceutically acceptable salt thereof is administered in Week 1 of a treatment cycle of 6 weeks. In some embodiment, Compound I or a pharmaceutically acceptable salt thereof is administered in Week 2 of a treatment cycle of 6 weeks. In some embodiment, Compound I or a pharmaceutically acceptable salt thereof is administered in Week 3 of a treatment cycle of 6 weeks. In some embodiment, Compound I or a pharmaceutically acceptable salt thereof is administered in Week 4 of a treatment cycle of 6 weeks. In some embodiment, Compound I or a pharmaceutically acceptable salt thereof is administered in Week 5 of a treatment cycle of 6 weeks. In some embodiment, Compound I or a pharmaceutically acceptable salt thereof is administered in Week 6 of a treatment cycle of 6 weeks.

In some embodiment, Compound I or a pharmaceutically acceptable salt thereof is administered in Week 1 of a treatment cycle of 9 weeks. In some embodiment, Compound I or a pharmaceutically acceptable salt thereof is administered in Week 2 of a treatment cycle of 9 weeks. In some embodiment, Compound I or a pharmaceutically acceptable salt thereof is administered in Week 3 of a treatment cycle of 9 weeks. In some embodiment, Compound I or a pharmaceutically acceptable salt thereof is administered in Week 3 of a treatment cycle of 9 weeks. In some embodiment, Compound I or a pharmaceutically acceptable salt thereof is administered in Week 4 of a treatment cycle of 9 weeks. In some embodiment, Compound I or a pharmaceutically acceptable salt thereof is administered in Week 5 of a treatment cycle of 9 weeks. In some embodiment, Compound I or a pharmaceutically acceptable salt thereof is administered in Week 6 of a treatment cycle of 9 weeks. In some embodiment, Compound I or a pharmaceutically acceptable salt thereof is administered in Week 7 of a treatment cycle of 9 weeks. In some embodiment, Compound I or a pharmaceutically acceptable salt thereof is administered in Week 8 of a treatment cycle of 9 weeks. In some embodiment, Compound I or a pharmaceutically acceptable salt thereof is administered in Week 9 of a treatment cycle of 9 weeks.

In some embodiments, Compound I or a pharmaceutically acceptable salt thereof is administered for 1, 2, 3, 4, 5, 6 or 7 consecutive days of the week in which Compound I or a pharmaceutically acceptable salt thereof is administered. In one embodiment, Compound I or a pharmaceutically acceptable salt thereof is administered on Days 1-2 of the week in which the Compound I or a pharmaceutically acceptable salt thereof is administered. In another embodiment, Compound I or a pharmaceutically acceptable salt thereof is administered on Days 1-3 of the week in which the Compound I or a pharmaceutically acceptable salt thereof is administered. In yet another embodiment, Compound I or a pharmaceutically acceptable salt thereof is administered on Days 1-4 of the week in which the Compound I or a pharmaceutically acceptable salt thereof is administered. In yet another embodiment, Compound I or a pharmaceutically acceptable salt thereof is administered on Days 1-5 of the week in which the Compound I or a pharmaceutically acceptable salt thereof is administered. In yet another embodiment, Compound I or a pharmaceutically acceptable salt thereof is administered on Days 1-6 of the week in which the Compound I or a pharmaceutically acceptable salt thereof is administered. In yet another embodiment, Compound I or a pharmaceutically acceptable salt thereof is administered on Days 1-7 of the week in which the Compound I or a pharmaceutically acceptable salt thereof is administered.

In the methods of treatment, prevention, or amelioration of one or more symptoms of the disorders, diseases, or conditions described herein, an appropriate dosage level of a PD-1 inhibitor (e.g., pembrolizumab) generally is ranging from about 0.1 to 2000 mg per day. In some embodiments, 1-500 mg once or multiple times per day is effective to obtain the desired results.

In certain embodiments, the PD-1 inhibitor (e.g., pembrolizumab) is administered once per day, twice per day, or three times per day. In certain embodiments, the PD-1 inhibitor (e.g., pembrolizumab) is administered once per day. In certain embodiments, the PD-1 inhibitor (e.g., pembrolizumab) is co-administered (e.g., in a single dosage form), once per day.

In certain embodiments, the PD-1 inhibitor (e.g., pembrolizumab) is administered once a week. In certain embodiments, the PD-1 inhibitor (e.g., pembrolizumab) is administered once every two, three, four or five weeks. In certain embodiments, the PD-1 inhibitor (e.g., pembrolizumab) is administered intravenously or through direct injection. In one preferred embodiment, the PD-1 inhibitor is administered every three weeks.

In some embodiments, the PD-1 inhibitor (e.g., pembrolizumab) is administered in Week 1 of a treatment cycle. In some embodiments, the PD-1 inhibitor (e.g., pembrolizumab) is administered in Week 2 of a treatment cycle. In some embodiments, the PD-1 inhibitor (e.g., pembrolizumab) is administered in Week 3 of a treatment cycle. In some embodiments, the PD-1 inhibitor (e.g., pembrolizumab) is administered in Week 4 of a treatment cycle. In some embodiments, the PD-1 inhibitor (e.g., pembrolizumab) is administered in Week 5 of a treatment cycle. In some embodiments, the PD-1 inhibitor (e.g., pembrolizumab) is administered in Week 6 of a treatment cycle. In some embodiments, the PD-1 inhibitor (e.g., pembrolizumab) is administered in Week 7 of a treatment cycle. In some embodiments, the PD-1 inhibitor (e.g., pembrolizumab) is administered in Week 8 of a treatment cycle. In some embodiments, the PD-1 inhibitor (e.g., pembrolizumab) is administered in Week 9 of a treatment cycle. In some embodiments, the treatment cycle is 3 weeks, 4 weeks, 5 weeks, 6 weeks, 7 weeks, 8 weeks, or 9 weeks.

In some embodiment, the PD-1 inhibitor (e.g., pembrolizumab) is administered in Week 1 of a treatment cycle of 3 weeks. In some embodiment, the PD-1 inhibitor (e.g., pembrolizumab) is administered in Week 2 of a treatment cycle of 3 weeks. In some embodiment, the PD-1 inhibitor (e.g., pembrolizumab) is administered in Week 3 of a treatment cycle of 3 weeks.

In some embodiment, the PD-1 inhibitor (e.g., pembrolizumab) is administered in Week 1 of a treatment cycle of 6 weeks. In some embodiment, the PD-1 inhibitor (e.g., pembrolizumab) is administered in Week 2 of a treatment cycle of 6 weeks. In some embodiment, the PD-1 inhibitor (e.g., pembrolizumab) is administered in Week 3 of a treatment cycle of 6 weeks. In some embodiment, the PD-1 inhibitor (e.g., pembrolizumab) is administered in Week 4 of a treatment cycle of 6 weeks. In some embodiment, the PD-1 inhibitor (e.g., pembrolizumab) is administered in Week 5 of a treatment cycle of 6 weeks. In some embodiment, the PD-1 inhibitor (e.g., pembrolizumab) is administered in Week 6 of a treatment cycle of 6 weeks.

In some embodiment, the PD-1 inhibitor (e.g., pembrolizumab) is administered in Week 1 of a treatment cycle of 9 weeks. In some embodiment, the PD-1 inhibitor (e.g., pembrolizumab) is administered in Week 2 of a treatment cycle of 9 weeks. In some embodiment, the PD-1 inhibitor (e.g., pembrolizumab) is administered in Week 3 of a treatment cycle of 9 weeks. In some embodiment, the PD-1 inhibitor (e.g., pembrolizumab) is administered in Week 4 of a treatment cycle of 9 weeks. In some embodiment, the PD-1 inhibitor (e.g., pembrolizumab) is administered in Week 5 of a treatment cycle of 9 weeks. In some embodiment, the PD-1 inhibitor (e.g., pembrolizumab) is administered in Week 6 of a treatment cycle of 9 weeks. In some embodiment, the PD-1 inhibitor (e.g., pembrolizumab) is administered in Week 7 of a treatment cycle of 9 weeks. In some embodiment, the PD-1 inhibitor (e.g., pembrolizumab) is administered in Week 8 of a treatment cycle of 9 weeks. In some embodiment, the PD-1 inhibitor (e.g., pembrolizumab) is administered in Week 9 of a treatment cycle of 9 weeks.

In some embodiments, the PD-1 inhibitor (e.g., pembrolizumab) is administered on Day 1, Day 2, Day 3, Day 4, Day 5, Day 6, or Day 7 of the week in which the PD-1 inhibitor (e.g., pembrolizumab) is administered. In one embodiment, the PD-1 inhibitor (e.g., pembrolizumab) is administered on Day 1 of the week in which the PD-1 inhibitor (e.g., pembrolizumab) is administered.

In certain embodiments, the PD-1 inhibitor is pembrolizumab and the amount of pembrolizumab that is administered is from about 10 mg/day up to, and including, 1000 mg/day. In certain embodiments, the amount of pembrolizumab that is administered is from about 10 mg/day to 600 mg/day. In certain embodiments, the amount of pembrolizumab that is administered is from about 100 mg/day to 600 mg/day. In certain embodiments, the amount of pembrolizumab that is administered per day is about 200 mg or about 400 mg. In some embodiments, pembrolizumab is administered at a dose of 200 mg once every 3 week (Q3W). In some embodiments, pembrolizumab is administered at a dose of 400 mg once every 6 week (Q6W).

In certain embodiments, the methods described herein further comprise administering pembrolizumab intravenously at a dosage of 1-10 mg/kg every three weeks. In certain embodiments, pembrolizumab is administered intravenously at a dosage of 2 mg/kg every three weeks. In certain embodiments, 2 mg/kg of pembrolizumab is administered as an intravenous infusion over 30 minutes every 3 weeks at 200 mg or every 6 weeks at 400 mg.

In some embodiments, Compound I or a pharmaceutically acceptable salt thereof and the PD-1 inhibitor (e.g., pembrolizumab) are not administered simultaneously, and instead the two compounds are administered at different times. In some embodiments, Compound I or a pharmaceutically acceptable salt thereof and the PD-1 inhibitor (e.g., pembrolizumab) are administered at least once during a treatment cycle. A treatment cycle as used herein is meant a period of time, during which each therapeutic agent has been administered at least once. A treatment cycle can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days. In some embodiments, a treatment cycle is 1, 2, 3, 4, 5, 6, 7, 8, 9, or 10 weeks. In certain embodiments, a treatment cycle is at least 3, 4, 5, 6, 7, 8, 9, 10, 11, or 12 weeks

It will be understood, however, that the specific dose level and frequency of dosage for any particular patient can be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy. The dosages can be determined by standard clinical techniques. The precise dose to be employed in the formulation will also depend on the route of administration, and the seriousness of a disease or condition, and in some embodiments, should be decided according to the judgment of the practitioner and each patient's circumstances.

The dose administered to a subject in the context of the present disclosure should be sufficient to affect a therapeutic response. One skilled in the art will recognize that dosage will depend upon a variety of factors including the potency of the specific compound, the age, condition and body weight of the patient, as well as the stage/severity of the disease. The dose will also be determined by the route (administration form) timing and frequency of administration.

### Additional Combination Therapy

In certain embodiments, the methods of combination therapy comprising Compound I or a pharmaceutically acceptable salt thereof and a PD-1 inhibitor (e.g., pembrolizumab) can also be combined or used in combination with a third agent or therapies useful in the treatment, prevention, or amelioration of one or more symptoms of a proliferative disorders, diseases, or conditions.

Suitable third agent of therapies include, but are not limited to, (1) alpha-adrenergic agents; (2) antiarrhythmic agents; (3) anti-atherosclerotic agents, such as ACAT inhibitors; (4) antibiotics, such as anthracyclines, bleomycins, mitomycin, dactinomycin, and plicamycin; (5) anticancer agents and cytotoxic agents, e.g., alkylating agents, such as nitrogen mustards, alkyl sulfonates, nitrosoureas, ethylenimines, and triazenes; (6) anticoagulants, such as acenocoumarol, argatroban, bivalirudin, lepirudin, fondaparinux, heparin, phenindione, warfarin, and xirnelagatran, (7) anti-diabetic agents, such as biguanides (e.g., metformin), glucosidase inhibitors (e.g., acarbose), insulins, meglitinides (e.g., repaglinide), sulfonylureas (e.g., glimepiride, glyburide, and glipizide), thiozolidinediones (e.g., troglitazone, rosiglitazone, and pioglitazone), and PPAR-gamma monists; (8) antifungal agents, such as amorolfine, amphotericin B, anidulafungin, bifonazole, butenafine, butoconazole, caspofungin, ciclopirox, clotrimazole, econazole, fenticonazole, filipin, fluconazole, isoconazole, itraconazole, ketoconazole, micafungin, miconazole, naftifine, natamycin, nystatin, oxyconazole, ravuconazole, posaconazole, rimocidin, sertaconazole, sulconazole, terbinafine, terconazole, tioconazole, and voriconazole; (9) antiinflammatories, e.g., non-steroidal anti-inflammatory agents, such as aceclofenac, acemetacin, amoxiprin, aspirin, azapropazone, benorilate, bromfenac, carprofen, celecoxib, choline magnesium salicylate, diclofenac, diflunisal, etodolac, etoricoxib, faislamine, fenbufen, fenoprofen, flurbiprofen, ibuprofen, indometacin, ketoprofen, ketorolac, lornoxicam, loxoprofen, lumiracoxib, meclofenamic acid, mefenamic acid, meloxicam, metamizole, methyl salicylate, magnesium salicylate, nabumetone, naproxen, nimesulide, oxyphenbutazone, parecoxib, phenylbutazone, piroxicam, salicyl salicylate, sulindac, sulfinpyrazone, suprofen, tenoxicam, tiaprofenic acid, and tolmetin; (10) antimetabolites, such as folate antagonists, purine analogues, and pyrimidine analogues; (11) anti-platelet agents, such as GPIIb/IIIa blockers (e.g., abciximab, eptifibatide, and tirofiban), P2Y(AC) antagonists (e.g., clopidogrel, ticlopidine and CS-747), cilostazol, dipyridamole, and aspirin; (12) antiproliferatives, such as methotrexate, FK506 (tacrolimus), and mycophenolate mofetil; (13) anti-TNF antibodies or soluble TNF receptor, such as etanercept, rapamycin, and leflunimide; (14) aP2 inhibitors; (15) beta-adrenergic agents, such as carvedilol and metoprolol; (16) bile acid secjuestrants, such as questran; (17) calcium channel blockers, such as amlodipine besylate; (18) chemotherapeutic agents; (19) cyclooxygenase-2 (COX-2) inhibitors, such as celecoxib and rofecoxib; (20) cyclosporins; (21) cytotoxic drugs, such as azathioprine and cyclophosphamide; (22) diuretics, such as chlorothiazide, hydrochlorothiazide, flumethiazide, hydroflumethiazide, bendroflumethiazide, methylchlorothiazide, trichloromethiazide, polythiazide, benzothiazide, ethacrynic acid, ticrynafen, chlorthalidone, furosenide, muzolimine, bumetanide, triamterene, amiloride, and spironolactone; (23) endothelin converting enzyme (ECE) inhibitors, such as phosphoramidon; (24) enzymes, such as L-asparaginase; (25) Factor VIIa Inhibitors and Factor Xa Inhibitors; (26) famesyl-protein transferase inhibitors; (27) fibrates; (28) growth factor inhibitors, such as modulators of PDGF activity; (29) growth hormone secretagogues; (30) HMG CoA reductase inhibitors, such as pravastatin, lovastatin, atorvastatin, simvastatin, NK-104 (a.k.a. itavastatin, nisvastatin, or nisbastatin), and ZD-4522 (also known as rosuvastatin, atavastatin, or visastatin); neutral endopeptidase (NEP) inhibitors; (31) hormonal agents, such as glucocorticoids (e.g., cortisone), estrogens/antiestrogens, androgens/antiandrogens, progestins, and luteinizing hormone-releasing hormone antagonists, and octreotide acetate; (32) immunosuppressants; (33) mineralocorticoid receptor antagonists, such as spironolactone and eplerenone; (34) microtubule-disruptor agents, such as ecteinascidins; (35) microtubule-stabilizing agents, such as pacitaxel, docetaxel, and epothilones A-F; (36) MTP Inhibitors; (37) niacin; (38) phosphodiesterase inhibitors, such as PDE III inhibitors (e.g., cilostazol) and PDE V inhibitors (e.g., sildenafil, tadalafil, and vardenafil); (39) plant-derived products, such as vinca alkaloids, epipodophyllotoxins, and taxanes; (40) platelet activating factor (PAF) antagonists; (41) platinum coordination complexes, such as cisplatin, satraplatin, and carboplatin; (42) potassium channel openers; (43) prenyl-protein transferase inhibitors; (44) protein tyrosine kinase inhibitors; (45) renin inhibitors; (46) squalene synthetase inhibitors; (47) steroids, such as aldosterone, beclometasone, betamethasone, deoxycorticosterone acetate, fludrocortisone, hydrocortisone (cortisol), prednisolone, prednisone, methylprednisolone, dexamethasone, and triamcinolone; (48) TNF-alpha inhibitors, such as tenidap; (49) thrombin inhibitors, such as hirudin; (50) thrombolytic agents, such as anistreplase, reteplase, tenecteplase, tissue plasminogen activator (tPA), recombinant tPA, streptokinase, urokinase, prourokinase, and anisoylated plasminogen streptokinase activator complex (APSAC); (51) thromboxane receptor antagonists, such as ifetroban; (52) topoisomerase inhibitors; (53) vasopeptidase inhibitors (dual NEP-ACE inhibitors), such as omapatrilat and gemopatrilat, and (54) other miscellaneous agents, such as, hydroxyurea, procarbazine, mitotane, hexamethylmelamine, and gold compounds.

In certain embodiments, the third therapies that may be used in combination with the methods provided herein include, but are not limited to, surgery, endocrine therapy, biologic response modifiers (e.g., interferons, interleukins, and tumor necrosis factor (TNF)), hyperthermia and cryotherapy, and agents to attenuate any adverse effects (e.g., antiemetics).

In certain embodiments, the third therapeutic agents that may be used in combination with the compounds provided herein include, but are not limited to, alkylating drugs (mechlorethamine, chlorambucil, cyclophosphamide, melphalan, and ifosfamide), antimetabolites (cytarabine (also known as cytosine arabinoside or Ara-C), and methotrexate), purine antagonists and pyrimidine antagonists (6-mercaptopurine, 5-fluorouracil, cytarbine, and gemcitabine), spindle poisons (vinblastine, vincristine, and vinorelbine), podophyllotoxins (etoposide, irinotecan, and topotecan), antibiotics (daunorubicin, doxorubicin, bleomycin, and mitomycin), nitrosoureas (carmustine and lomustine), enzymes (asparaginasc), and hormones (tamoxifen, leuprolide, flutamide, and megestrol), imatinib, adriamycin, dexamethasone, and cyclophosphamide. For a more comprehensive discussion of updated cancer therapies; See, http://www.nci.nih.gov/, a list of the FDA approved oncology drugs at http://www.fda.gov/cder/cancer/dniglistframe.htm, and The Merck Manual, Seventeenth Ed. 1999, the entire contents of which are hereby incorporated by reference.

In another embodiment, methods provided herein comprise administration of Compound I or a pharmaceutically acceptable salt thereof and a PD-1 inhibitor (e.g., pembrolizumab), together with administration of one or more chemotherapeutic agents and/or therapies selected from: alkylation agents (e.g., cisplatin, carboplatin); antimetabolites (e.g., methotrexate and 5-FU); antitumor antibiotics (e.g., adriamymycin and bleomycin); antitumor vegetable alkaloids (e.g., taxol and etoposide); antitumor hormones (e.g., dexamethasone and tamoxifen); antitumor immunological agents (e.g., interferon α, β, and γ); radiation therapy; and surgery. In certain embodiments, the one or more chemotherapeutic agents and/or therapies are administered to the subject before, during, or after the administration of Compound I or a pharmaceutically acceptable salt thereof and the PD-1 inhibitor (e.g., pembrolizumab).

Such other agents, or drugs, can be administered, by a route and in an amount commonly used therefor, simultaneously or sequentially with Compound I or a pharmaceutically acceptable salt thereof and the PD-1 inhibitor (e.g., pembrolizumab). When Compound I or a pharmaceutically acceptable salt thereof and the PD-1 inhibitor (e.g., pembrolizumab) are used contemporaneously with one or more other drugs, a pharmaceutical composition containing such other drugs in addition to Compound I or a pharmaceutically acceptable salt thereof and the PD-1 inhibitor (e.g., pembrolizumab) can be utilized, but is not required. Accordingly, the pharmaceutical compositions provided herein include those that also contain one or more other active ingredients or therapeutic agents, in addition to Compound I or a pharmaceutically acceptable salt thereof.

### Pharmaceutical Compositions and Routes of Administration

Provided herein are a pharmaceutical composition comprising Compound I or a pharmaceutically acceptable salt thereof, the PD-1 inhibitor (e.g., pembrolizumab), and a pharmaceutically acceptable excipient, adjuvant, carrier, buffer, or stabilizer. In some embodiments, Compound I or a pharmaceutically acceptable salt thereof and the PD-1 inhibitor (e.g., pembrolizumab) are present in the same pharmaceutical composition. In some embodiments, Compound I or a pharmaceutically acceptable salt thereof and the PD-1 inhibitor (e.g., pembrolizumab) in different pharmaceutical compositions.

In some embodiment, a first pharmaceutical composition comprises Compound I or a pharmaceutically acceptable salt thereof and a first pharmaceutically acceptable excipient or carrier. In some embodiment, a second pharmaceutical composition comprises the PD-1 inhibitor (e.g., pembrolizumab) and a second pharmaceutically acceptable excipient or carrier.

In one embodiment, the pharmaceutical compositions are provided in a dosage form for oral administration, which comprise a compound provided herein, and one or more pharmaceutically acceptable excipients or carriers. The pharmaceutical compositions provided herein that are formulated for oral administration may be in tablet, capsule, powder, or liquid form. In some embodiments, a tablet comprises a solid carrier or an adjuvant. Liquid pharmaceutical compositions generally comprise a liquid carrier such as water, petroleum, animal or vegetable oils, mineral oil, or synthetic oil. Physiological saline solution, dextrose or other saccharide solution, or glycols such as ethylene glycol, propylene glycol, or polyethylene glycol may be included. In some embodiments, a capsule comprises a solid carrier such as gelatin.

In another embodiment, the pharmaceutical compositions are provided in a dosage form for parenteral administration, which comprise a compound provided herein, and one or more pharmaceutically acceptable excipients or carriers. Where pharmaceutical compositions may be formulated for intravenous, cutaneous or subcutaneous injection, the active ingredient will be in the form of a parenterally acceptable aqueous solution, which is pyrogen-free and has a suitable pH, isotonicity, and stability. Those of relevant skill in the art are well able to prepare suitable solutions using, for example, isotonic vehicles, such as Sodium Chloride injection, Ringer's injection, or Lactated Ringer's injection. In some embodiments, preservatives, stabilizers, buffers, antioxidants, and/or other additives are included as required.

In yet another embodiment, the pharmaceutical compositions are provided in a dosage form for topical administration, which comprise a compound provided herein, and one or more pharmaceutically acceptable excipients or carriers.

The pharmaceutical compositions can also be formulated as modified release dosage forms, including delayed-, extended-, prolonged-, sustained-, pulsatile-, controlled-, accelerated-, fast-, targeted-, and programmed-release, and gastric retention dosage forms. These dosage forms can be prepared according to conventional methods and techniques known to those skilled in the art (see, Remington: The Science and Practice of Pharmacy, supra; Modified-Release Drug Delivery Technology, 2nd Edition, Rathbone et al., Eds., Marcel Dekker, Inc.: New York, NY, 2008).

The pharmaceutical compositions provided herein can be provided in a unit-dosage form or multiple-dosage form. A unit-dosage form, as used herein, refers to physically discrete a unit suitable for administration to a human and animal subject, and packaged individually as is known in the art. Each unit-dose contains a predetermined quantity of an active ingredient(s) sufficient to produce the desired therapeutic effect, in association with the required pharmaceutical carriers or excipients. Examples of a unit-dosage form include an ampoule, syringe, and individually packaged tablet and capsule. A unit-dosage form may be administered in fractions or multiples thereof. A multiple-dosage form is a plurality of identical unit-dosage forms packaged in a single container to be administered in segregated unit-dosage form. Examples of a multiple-dosage form include a vial, bottle of tablets or capsules, or bottle of pints or gallons.

The pharmaceutical compositions provided herein can be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

In certain embodiments, the pharmaceutical compositions provided herein further comprise one or more chemotherapeutic agents as defined herein.

### A. Oral Administration

The pharmaceutical compositions provided herein for oral administration can be provided in solid, semisolid, or liquid dosage forms for oral administration. As used herein, oral administration also includes buccal, lingual, and sublingual administration. Suitable oral dosage forms include, but are not limited to, tablets, fastmelts, chewable tablets, capsules, pills, strips, troches, lozenges, pastilles, cachets, pellets, medicated chewing gum, bulk powders, effervescent or non-effervescent powders or granules, oral mists, solutions, emulsions, suspensions, wafers, sprinkles, elixirs, and syrups. In addition to the active ingredient(s), the pharmaceutical compositions can contain one or more pharmaceutically acceptable carriers or excipients, including, but not limited to, binders, fillers, diluents, disintegrants, wetting agents, lubricants, glidants, coloring agents, dye-migration inhibitors, sweetening agents, flavoring agents, emulsifying agents, suspending and dispersing agents, preservatives, solvents, non-aqueous liquids, organic acids, and sources of carbon dioxide.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, PA); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The amount of a binder or filler in the pharmaceutical compositions provided herein varies upon the type of formulation, and is readily discernible to those of ordinary skill in the art. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets. The amount of a diluent in the pharmaceutical compositions provided herein varies upon the type of formulation, and is readily discernible to those of ordinary skill in the art.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of a disintegrant in the pharmaceutical compositions provided herein varies upon the type of formulation, and is readily discernible to those of ordinary skill in the art. The amount of a disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W. R. Grace Co., Baltimore, MD) and CAB-O-SIL® (Cabot Co. of Boston, MA); and mixtures thereof. The pharmaceutical compositions provided herein may contain about 0.1 to about 5% by weight of a lubricant.

Suitable glidants include, but are not limited to, colloidal silicon dioxide, CAB-O-SIL® (Cabot Co. of Boston, MA), and asbestos-free talc. Suitable coloring agents include, but are not limited to, any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Suitable flavoring agents include, but are not limited to, natural flavors extracted from plants, such as fruits, and synthetic blends of compounds which produce a pleasant taste sensation, such as peppermint and methyl salicylate. Suitable sweetening agents include, but are not limited to, sucrose, lactose, mannitol, syrups, glycerin, and artificial sweeteners, such as saccharin and aspartame. Suitable emulsifying agents include, but are not limited to, gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suitable suspending and dispersing agents include, but are not limited to, sodium carboxymethylcellulose, pectin, tragacanth, Veegum, acacia, sodium carbomethylcellulose, hydroxypropyl methylcellulose, and polyvinylpyrrolidone. Suitable preservatives include, but are not limited to, glycerin, methyl and propylparaben, benzoic add, sodium benzoate and alcohol. Suitable wetting agents include, but are not limited to, propylene glycol monostearate, sorbitan monooleate, diethylene glycol monolaurate, and polyoxyethylene lauryl ether. Suitable solvents include, but are not limited to, glycerin, sorbitol, ethyl alcohol, and syrup. Suitable non-aqueous liquids utilized in emulsions include, but are not limited to, mineral oil and cottonseed oil. Suitable organic acids include, but are not limited to, citric and tartaric acid. Suitable sources of carbon dioxide include, but are not limited to, sodium bicarbonate and sodium carbonate.

It should be understood that many carriers and excipients may serve several functions, even within the same formulation.

The pharmaceutical compositions provided herein for oral administration can be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenyl salicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms can be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein for oral administration can be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

The pharmaceutical compositions provided herein for oral administration can be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquid or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde, e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or poly-alkylene glycol, including, 1,2-dimetboxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations can further comprise one or more antioxidants, such as butylated hydroxytoluene (BHT), butylated hydroxyanisole (BHA), propyl gallate, vitamin E, hydroquinone, hydroxycoumarins, ethanolarnine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions provided herein for oral administration can be also provided in the forms of liposomes, micelles, microspheres, or nanosystems. Micellar dosage forms can be prepared as described in U.S. Pat. No. 6,350,458.

The pharmaceutical compositions provided herein for oral administration can be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms.

The pharmaceutical compositions provided herein for oral administration can be formulated as immediate or modified release dosage forms, including delayed-, sustained, pulsed-, controlled, targeted-, and programmed-release forms.

### B. Parenteral Administration

The pharmaceutical compositions provided herein can be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, intravesical, and subcutaneous administration.

The pharmaceutical compositions provided herein for parenteral administration can be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration can include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Suitable non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Suitable water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

Suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride.

Suitable suspending and dispersing agents are those as described herein, including sodium carboxymethylcelluose, hydroxypropyl methylcellulose, and polyvinylpyrrolidone. Suitable emulsifying agents are those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, KS).

When the pharmaceutical compositions provided herein are formulated for multiple dosage administration, the multiple dosage parenteral formulations must contain an antimicrobial agent at bacteriostatic or fungi static concentrations. All parenteral formulations must be sterile, as known and practiced in the art.

In one embodiment, the pharmaceutical compositions for parenteral administration are provided as ready-to-use sterile solutions. In another embodiment, the pharmaceutical compositions are provided as sterile dry soluble products, including lyophilized powders and hypodermic tablets, to be reconstituted with a vehicle prior to use. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

The pharmaceutical compositions provided herein for parenteral administration can be formulated as immediate or modified release dosage forms, including delayed-, sustained, pulsed-, controlled, targeted-, and programmed-release forms.

The pharmaceutical compositions provided herein for parenteral administration can be formulated as a suspension, solid, semi-solid, or thixotropic liquid, for administration as an implanted depot. In one embodiment, the pharmaceutical compositions provided herein are dispersed in a solid inner matrix, which is surrounded by an outer polymeric membrane that is insoluble in body fluids but allows the active ingredient in the pharmaceutical compositions diffuse through.

Suitable inner matrixes include, but are not limited to, polymethylmethacrylate, polybutyl-methacrylate, plasticized or unplasticized polyvinylchloride, plasticized nylon, plasticized polyethylene terephthalate, natural rubber, polyisoprene, polyisobutylene, polybutadiene, polyethylene, ethylene-vinyl acetate copolymers, silicone rubbers, polydimethylsiloxanes, silicone carbonate copolymers, hydrophilic polymers, such as hydrogels of esters of acrylic and metliacrylic acid, collagen, cross-linked polyvinyl alcohol, and cross-linked partially hydrolyzed polyvinyl acetate.

Suitable outer polymeric membranes include but are not limited to, polyethylene, polypropylene, ethylene/propylene copolymers, ethylene/ethyl acrylate copolymers, ethylene/vinyl acetate copolymers, silicone rubbers, polydimethyl siloxanes, neoprene rubber, chlorinated polyethylene, polyvinylchloride, vinyl chloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

### C. Topical Administration

The pharmaceutical compositions provided herein can be administered topically to the skin, orifices, or mucosa. The topical administration, as used herein, includes (intra)dermal, conjunctival, intracorneal, intraocular, ophthalmic, auricular, transdermal, nasal, vaginal, urethral, respiratory, and rectal administration.

The pharmaceutical compositions provided herein can be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, and dermal patches. The topical formulation of the pharmaceutical compositions provided herein can also comprise liposomes, micelles, microspheres, nanosystems, and mixtures thereof.

Pharmaceutically acceptable carriers and excipients suitable for use in the topical formulations provided herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The pharmaceutical compositions can also be administered topically by electroporation, iontophoresis, phonophoresis, sonophoresis, or microneedle or needle-free injection, such as POWDERJECT™ (Chiron Corp., Emeryville, CA), and BIOJECT™ (Bioject Medical Technologies Inc., Tualatin, OR).

The pharmaceutical compositions provided herein can be provided in the forms of ointments, creams, and gels. Suitable ointment vehicles include oleaginous or hydrocarbon vehicles, including lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (see, Remington: The Science and Practice of Pharmacy, supra). These vehicles are emollient but generally require addition of antioxidants and preservatives.

Suitable cream base can be oil-in-water or water-in-oil. Suitable cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. In some embodiments, the emulsifier in a cream formulation is a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include, but are not limited to, crosslinked acrylic acid polymers, such as carbomers, carboxypolyalkylenes, and CARBOPOL®; hydrophilic polymers, such as polyethylene oxides, polyoxyethylene-polyoxypropylene copolymers, and polyvinyl alcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions provided herein can be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with the pharmaceutical compositions provided herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, and hydrogels, such as polyvinyl alcohol, hydroxyethyl methacrylate, and polyacrylic acid. Combinations of the various vehicles can also be used. Rectal and vaginal suppositories may be prepared by compressing or molding. The typical weight of a rectal and vaginal suppository is about 2 to about 3 g.

The pharmaceutical compositions provided herein can be administered ophthalmically in the forms of solutions, suspensions, ointments, emulsions, gel-forming solutions, powders for solutions, gels, ocular inserts, and implants.

The pharmaceutical compositions provided herein can be administered intranasally or by inhalation to the respiratory tract. The pharmaceutical compositions can be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions can also be provided as a dry powder for insufflation, alone or in combination with an inert carrier such as lactose or phospholipids; and nasal drops. For intranasal use, the powder can comprise a bioadhesive agent, including chitosan or cyclodextrin.

Solutions or suspensions for use in a pressurized container, pump, spray, atomizer, or nebulizer can be formulated to contain ethanol, aqueous ethanol, or a suitable alternative agent for dispersing, solubilizing, or extending release of the active ingredient provided herein; a propellant as solvent; and/or a surfactant, such as sorbitan trioleate, oleic acid, or an oligolactic acid.

The pharmaceutical compositions provided herein can be micronized to a size suitable for delivery by inhalation, such as about 50 micrometers or less, or about 10 micrometers or less. Particles of such sizes can be prepared using a comminuting method known to those skilled in the art, such as spiral jet milling, fluid bed jet milling, supercritical fluid processing to form nanoparticles, high pressure homogenization, or spray drying.

Capsules, blisters, and cartridges for use in an inhaler or insufflator can be formulated to contain a powder mix of the pharmaceutical compositions provided herein; a suitable powder base, such as lactose or starch; and a performance modifier, such as l-leucine, mannitol, or magnesium stearate. The lactose may be anhydrous or in the form of the monohydrate. Other suitable excipients or carriers include, but are not limited to, dextran, glucose, maltose, sorbitol, xylitol, fructose, sucrose, and trehalose. The pharmaceutical compositions provided herein for inhaled/intranasal administration can further comprise a suitable flavor, such as menthol and levomenthol; and/or sweeteners, such as saccharin and saccharin sodium.

The pharmaceutical compositions provided herein for topical administration can be formulated to be immediate release or modified release, including delayed-, sustained-, pulsed-, controlled-, targeted, and programmed release.

### D. Modified Release

The pharmaceutical compositions provided herein can be formulated as a modified release dosage form. As used herein, the term “modified release” refers to a dosage form in which the rate or place of release of the active ingredient(s) is different from that of an immediate dosage form when administered by the same route. Modified release dosage forms include, but are not limited to, delayed-, extended-, prolonged-, sustained-, pulsatile-, controlled-, accelerated- and fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphism of the active ingredient(s).

Examples of modified release include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; and 6,699,500.

### 1. Matrix Controlled Release Devices

The pharmaceutical compositions provided herein in a modified release dosage form can be fabricated using a matrix controlled release device known to those skilled in the art (see, Takada et al. in “Encyclopedia of Controlled Drug Delivery,” Vol. 2, Mathiowitz Ed., Wiley, 1999).

In certain embodiments, the pharmaceutical compositions provided herein in a modified release dosage form is formulated using an erodible matrix device, which is water-swellable, erodible, or soluble polymers, including, but not limited to, synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethyl hydroxyethyl cellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, NJ); poly(2-hydroxyethyl-methacrylate), polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methyl methacrylate, ethyl methacrylate, ethylacrylate, (2-dimethylaminoethyl)methacrylate, and (trimethylaminoethyl)methacrylate chloride.

In certain embodiments, the pharmaceutical compositions provided herein are formulated with a non-erodible matrix device. The active ingredient(s) is dissolved or dispersed in an inert matrix and is released primarily by diffusion through the inert matrix once administered. Materials suitable for use as a non-erodible matrix device include, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinyl acetate copolymers, ethylene/propylene copolymers, ethylene/ethyl acrylate copolymers, vinyl chloride copolymers with vinyl acetate, vinylidene chloride, ethylene and propylene, ionomer polyethylene terephthalate, butyl rubbers, epichlorohydrin rubbers, ethylene/vinyl alcohol copolymer, ethylene/vinyl acetate/vinyl alcohol terpolymer, ethylene/vinyloxyethanol copolymer, polyvinyl chloride, plasticized nylon, plasticized polyethylene terephthalate, natural rubber, silicone rubbers, polydimethylsiloxanes, and silicone carbonate copolymers; hydrophilic polymers, such as ethyl cellulose, cellulose acetate, crospovidone, and cross-linked partially hydrolyzed polyvinyl acetate; and fatty compounds, such as carnauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients or carriers in the compositions.

The pharmaceutical compositions provided herein in a modified release dosage form can be prepared by methods known to those skilled in the art, including direct compression, dry or wet granulation followed by compression, and melt-granulation followed by compression.

### Articles of Manufacture

The compounds provided herein can also be provided as an article of manufacture using packaging materials well known to those of skill in the art. See, e.g., U.S. Pat. Nos. 5,323,907; 5,052,558; and 5,033,252. Examples of pharmaceutical packaging materials include, but are not limited to, blister packs, bottles, tubes, inhalers, pumps, bags, vials, containers, syringes, and any packaging material suitable for a selected formulation and intended mode of administration and treatment.

Provided herein also are kits which, when used by the medical practitioner, can simplify the administration of appropriate amounts of active ingredients to a subject. In certain embodiments, the kit provided herein includes one or more containers and a dosage form of Compound I or a pharmaceutically acceptable salt thereof and a PD-1 inhibitor (e.g., pembrolizumab).

In certain embodiments, the kit provided herein includes one or more containers and a dosage form of Compound I or a pharmaceutically acceptable salt thereof and PD-1 inhibitor (e.g., pembrolizumab). Kits provided herein can further include devices that are used to administer the active ingredients. Examples of such devices include, but are not limited to, syringes, needle-less injectors drip bags, patches, and inhalers.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, Water for Injection USP, Sodium Chloride Injection, Ringer's Injection, Dextrose Injection, Dextrose and Sodium Chloride Injection, and Lactated Ringer's Injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

### EXAMPLES

As used herein, the symbols and conventions used in these processes, schemes and examples, regardless of whether a particular abbreviation is specifically defined, are consistent with those used in the contemporary scientific literature, for example, the Journal of the American Chemical Society or the Journal of Biological Chemistry. Specifically, but without limitation, the following abbreviations may be used in the examples and throughout the specification: g (grams); mg (milligrams); mL (milliliters); μL, (microliters); M (molar); mM (millimolar), μM (micro molar); eq. (equivalent); mmol (millimoles), Hz (Hertz), MHz (megahertz); hr or hrs (hour or hours); min (minutes); and MS (mass spectrometry).

For all of the following examples, standard work-up and purification methods known to those skilled in the art can be utilized. Unless otherwise indicated, all temperatures are expressed in ° C. (degrees Centigrade). All reactions conducted at room temperature unless otherwise noted. Synthetic methodologies illustrated herein are intended to exemplify the applicable chemistry through the use of specific examples and are not indicative of the scope of the disclosure.

### Example 1: Synthesis of 4-(2-(difluoromethyl)-1H-benzo[d]imidazol-1-yl)-N-(2-methyl-1-(2-(1-methylpiperidin-4-yl)phenyl)propan-2-yl)-6-morpholino-1,3,5-triazin-2-amine, Compound I

Synthesis of Compound I is described in U.S. Pat. No. 9,056,852 B2, which is incorporated by reference for such disclosure.

A mixture of 4-(2-(difluoromethyl)-1H-benzo[d]imidazol-1-yl)-N-(2-methyl-1-(2-(piperidin-4-yl)phenyl)propan-2-yl)-6-morpholino-1,3,5-triazin-2-amine (80 mg, 0.14 mmol), aq. formaldehyde (37%, 23 mg), and sodium cyanoborohydride (11 mg, 0.17 mmol) in methanol (2 mL) was stirred at room temperature for 1 hr. The crude product was purified by prep-HPLC to give compound I (11 mg, 13% yield) as a white solid: 99% purity (HPLC); MS m z: 577.3 (M+1); ¹H NMR (CDCl₃, 500 MHz) δ 8.37 (d, 1H), 7.90 (d, 1H), 7.64 (t, 1H), 7.42 (m, 2H), 7.32 (d, 1H), 7.24 (1, 1H), 7.13 (t, 1H), 7.07 (d, 1H), 5.15 (s, 1H), 4.00-3.70 (m, 8H), 3.28 (s, 2H), 2.94 (m, 2H), 2.78 (m, 2H), 2.28 (s, 3H), 1.891.60 (m, 6H), 1.53 (s, 6H) ppm.

### Example 2: MC38 Murine Colon Carcinoma Model for Combination of PI3K Inhibitor and PD-1 Inhibitor

The combination of a PI3K inhibitor (Compound I) and a PD-1 inhibitor (RMP1-14) was evaluated utilizing a MC38 murine colon carcinoma syngeneic model in female immunocompetent C57BL/6 mice. The study was designed to plot the survival time of murine specimens when treated with the combination of Compound I and the PD-1 inhibitor.

Compound I dosing solutions were prepared weekly. Formulations of Compound I were prepared by adding the appropriate amount of 20% VE-TPGS, 80% 100 mM citrate buffer, pH 3.5 solution (vehicle) to Compound I. The resulting suspension was stirred overnight to yield a 5 mg/mL dose solution. This solution provided a dosage of 50 mg/kg in a dosing volume of 10 mL/kg. The Compound I dosing solution was stored at 4° C. when not in use.

The PD-1 inhibitor (anti-PD-1 antibody Clone RMP1-14, Lot No. 5792-599016J1) was purchased by CR Discovery Services from Bio X Cell and stored at 4° C. upon receipt. Anti-PD-1 antibody dosing solutions were prepared by diluting aliquots of the stock (6.37 mg/mL) to 0.5 mg/mL in sterile PBS, yielding a dosage of 100 g/animal in a fixed dosing volume of 0.2 mL/animal.

Tumors were measured twice weekly. Treatment response was to be evaluated from tumor growth delay (TGD), the increase in median time-to-endpoint (TTE) in treated versus control mice, and from comparison of survival curves using logrank analysis. Each animal was marked for tumor progression when its tumor reached the 1000 mm³ volume endpoint, and any animal that did not reach the endpoint was euthanized at the end of the study and assigned a TTE value equal to the last day of the study (Day 45).

The median TTE for control Group 1 was 19.0 days, establishing a maximum TGD of 137% for this study. All treatments evaluated in the study were acceptably-tolerated, with acceptable mean BW losses and no treatment-related (TR) deaths.

### Tumor Growth Delay (TGD) Analysis

Animals were monitored individually for tumor growth until Day 45. The study protocol specified a tumor growth delay assay based on the median time to endpoint (TTE) in a treated group versus the control group. Each animal was marked for tumor progression (TP) when its tumor reached the 1000 mm³ volume endpoint. The TTE for each mouse was calculated with the following equation:

$$TTE = \frac{{log}_{10} ( endpoint volume ) - b}{m}$$

where b is the intercept and m is the slope of the line obtained by linear regression of a log-transformed tumor growth data set. The data set is comprised of the first observation that exceeded the study endpoint volume and the three consecutive observations that immediately preceded the attainment of the endpoint volume. Any animal that did not reach endpoint was euthanized at the end of the study and assigned a TTE value equal to the last day of the study (Day 45). In instances in which the log-transformed calculated TTE preceded the day prior to reaching endpoint or exceeded the day of reaching tumor volume endpoint, a linear interpolation was performed to approximate TTE.

On Day 45, MTV (n) was defined as the median tumor volume of the number of animals, n, that survived to the last day and whose tumors had not reached the volume endpoint. Any animal determined to have died from TR causes was to be assigned a TTE value equal to the day of death. Any animal that died from NTR causes was to be excluded from the analysis. Treatment outcome was evaluated from TGD, which was defined as the increase in the median TTE for a treatment group compared to the control group:

$$TGD = T - C$$

expressed in days, or as a percentage of the median TTE of the control group:

$$\% TGD = \frac{T - C}{C} \times 100$$

where
T=median TTE for a treatment group,
C=median TTE for the control group.

Survival was analyzed by the Kaplan-Meier method. The logrank (Mantel-Cox) and Gehan-Breslow-Wilcoxon tests determined the significance of the difference between the overall survival experiences (survival curves) of two groups, based on TTE values. A scatter plot was constructed to show TTE values for individual mice, by group. Group median tumor volumes were plotted as functions of time. When an animal exited the study because of tumor size, the final tumor volume recorded for the animal was included with the data used to calculate the median volume at subsequent time points. A Kaplan-Meier plot was constructed to show the percentage of animals in each group remaining on study versus time.

### Example 2a

On Day 1 of the study, four groups of C57BL/6 mice (n=10 for control group and PD-1 monotherapy; n=20 for combination therapy) began dosing. Compound I was administered by oral gavage (p.o.) at a dosage of 50 mg/kg in a dosing volume of 10 mL/kg and was adjusted according to the most recent body weight measurement. The PD-1 inhibitor was administered intraperitoneally (i.p.) at a dosage of 100 g/animal in a fixed dosing volume of 0.2 mL/animal.

Group 1 was left untreated and served as the control group for the % TGD.

Group 2 was treated with the PD-1 inhibitor i.p. at a dosage of 100 g/animal twice weekly for two weeks.

Group 3 received Compound I p.o. at 50 mg/kg qd×7 starting on Day 1. Beginning on Day 8, this group was then treated with the PD-1 inhibitor i.p. at 100 g/animal biwk×2.

Group 4 received Compound I p.o. at 50 mg/kg qd×7 starting on Day 1. Beginning on Day 8, this group was treated with the PD-1 inhibitor i.p. at 100 g/animal biwk×2 with the final dose occurring on Day 18. On Day 22, treatment with Compound I resumed at 50 mg/kg once daily for five days (qd×5, start Day 22).

Group 5 received Compound I p.o. at 50 mg/kg qd×7 starting on Day 1. On Day 8, these animals received the PD-1 inhibitor i.p. at 100 g/animal biwk×2 with the final dose occurring on Day 18. A second course of the PD-1 inhibitor was administered biwk×2 beginning on Day 27.

Group 6 received Compound I p.o. at 50 mg/kg qd×7 starting on Day 1. On Day 8, these animals received the PD-1 inhibitor i.p. at 100 g/animal biwk×2 with the final dose occurring on Day 18. On Day 22, treatment with Compound I resumed at 50 mg/kg qd×5. A second course of the PD-1 inhibitor was administered biwk×2 beginning on Day 27.

Group 7 received Compound I p.o. at 50 mg/kg qd×7, start D1, and then again for seven days starting on Day 22 (qd×7, start Day 22) combined with the PD-1 inhibitor i.p. at 100 g/animal biwk×2, start Day 8.

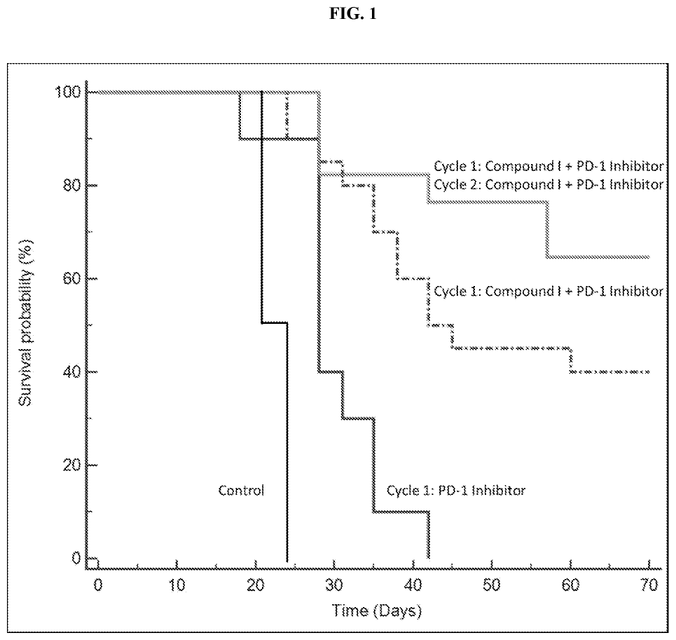

Treatment with Compound I and the PD-1 inhibitor was well-tolerated. All of the Compound I/PD-1 inhibitor combinations provided statistically significant survival benefits. Combination regimens that administered Compound I and the PD-1 inhibitor in two treatment cycles were more effective than one treatment cycle (Group 6 vs Group 3) and more effective than a single cycle of the PD-1 inhibitor (Group 6 vs Group 2). The results are depicted in FIG. 1.

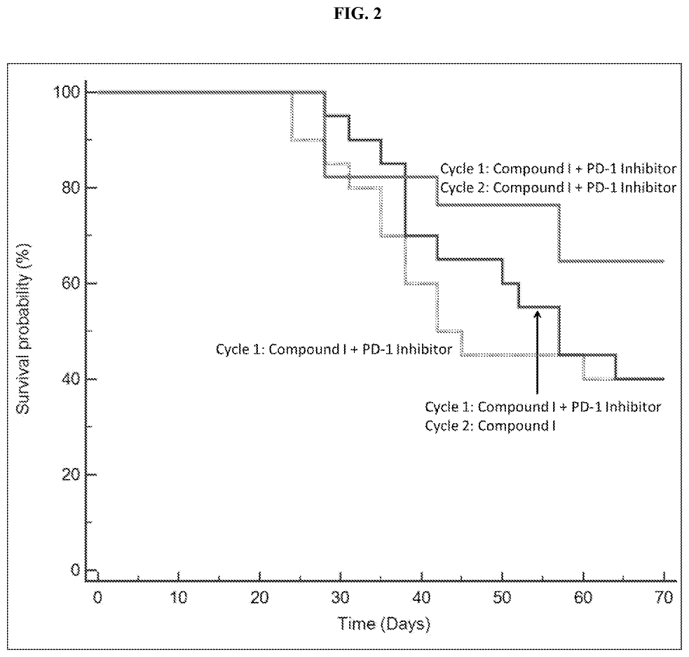

Combination regimens that administered Compound I and the PD-1 inhibitor in two treatment cycles were more effective than the administration of one combination treatment cycle and one Compound I treatment cycle (Group 6 vs Group 4) and more effective than the administration of a single cycle of a combination treatment with Compound I and the PD-1 inhibitor. (Group 6 vs Group 3). The results are depicted in FIG. 2.

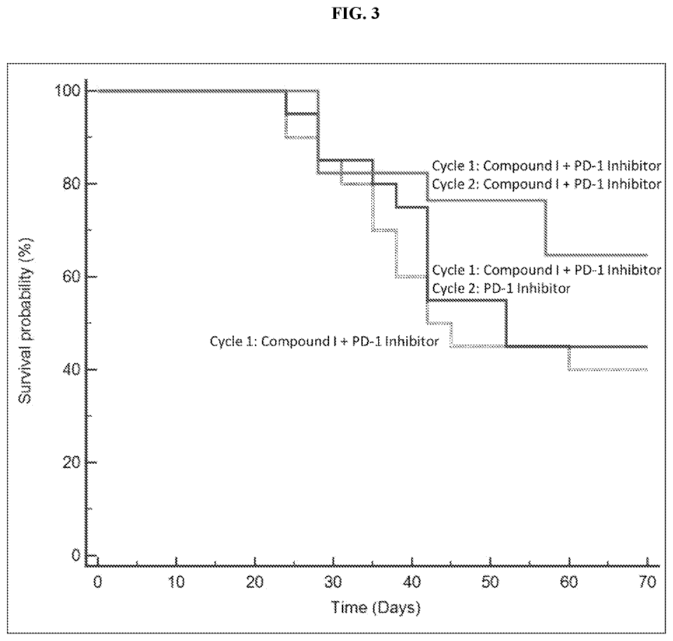

Combination regimens that administered Compound I and the PD-1 inhibitor in two treatment cycles were more effective than the administration one combination cycle and one PD-1 inhibitor treatment cycle (Group 6 vs Group 5) and more effective than the administration of a single cycle of a combination treatment with Compound I and the PD-1 inhibitor. (Group 6 vs Group 3). The results are depicted in FIG. 3.

### Example 3: Clinical Study of Combination Treatment of Compound I and Pembrolizumab

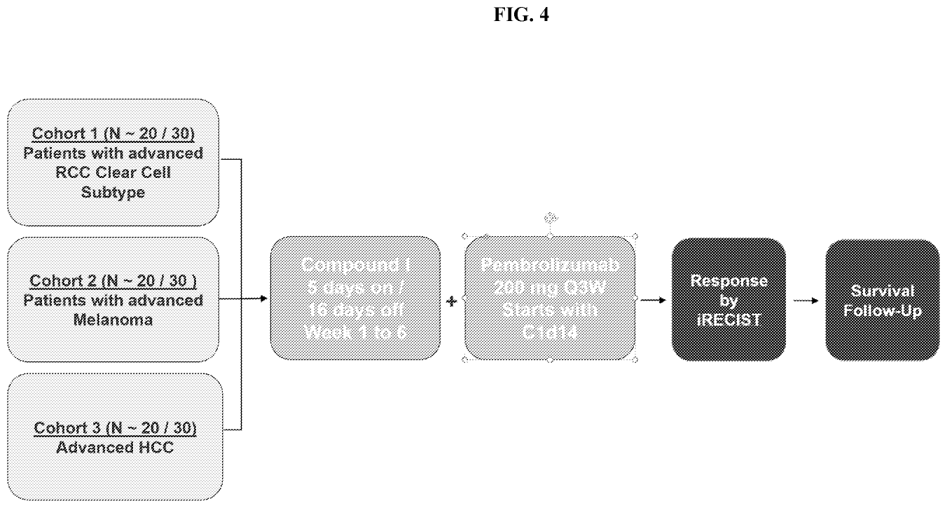

This example relates a 3-cohort clinical study of Compound I in combination with anti-PD-1 therapy with Pembrolizumab in subjects with advanced renal cell carcinoma, advanced melanoma, or advanced hepatocellular carcinoma. The study includes a DLT window of 42 days (6 weeks). The study design is described in FIG. 4.

### Study Design & Dosing Schedule

The study includes administering combination of Compound I and Pembrolizumab to three cohorts of patients (N=20 to 30): Cohort 1: patients with advanced renal cell carcinoma (RCC), in particular, advanced RCC Clear Cell Subtype; Cohort 2: patients with melanoma; and Cohort 3: patients with advanced hepatocellular carcinoma (HCC).

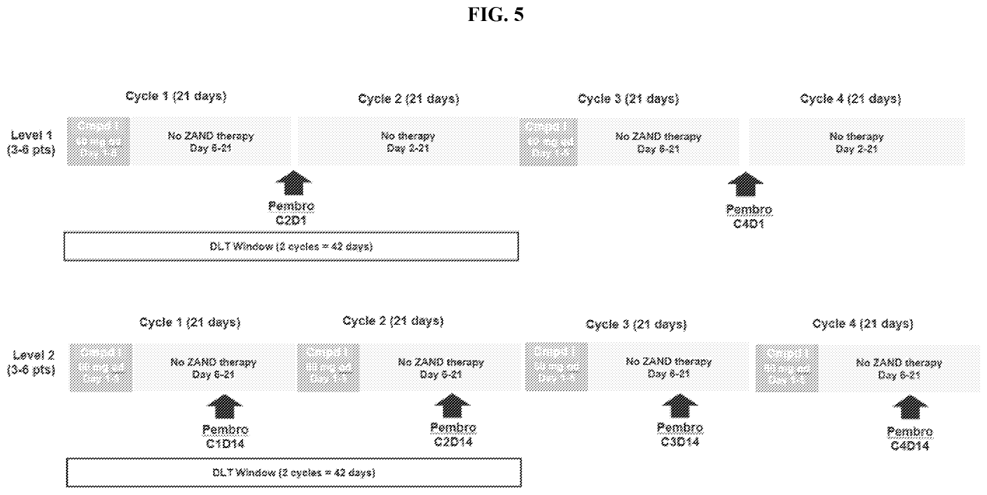

The dosing schedule of the study design is illustrated in FIG. 5.

In level 1 of the study, Compound I is administered for 5 days on Days 1 to 5, and off for 16 days in the first three weeks (Cycle 1), and not administered in Cycle 2. Compound I is administered once daily (qd) at a dose of 60 mg/day. Pembrolizumab is administered starting on Day 1 of Week 4 (Day 1 of Cycle 2; C2D1), and not administered in Cycle 3 in which Compound I is administered. Pembrolizumab is administered via infusion at a dose of 400 mg. Then the administration of Compound I repeats on Cycles 3, 5, 7, . . . and the administration of Pembrolizumab repeats on Cycles of 4, 6, 8, . . . . Each of the Cycle is 3 weeks (21 days).

In level 2 of the study, Compound I is administered for 5 days on Days 1 to 5, and off for 16 days in each of the 3-week treatment cycles once daily (qd) at a dose of 60 mg/day. Pembrolizumab is administered on Day 14 of each of the 3-week treatment cycles once via infusion at a dose of 400 mg. Then the administration of Compound I and Pembrolizumab repeats on Cycles of 2, 3, 4, 5, 7, 8, . . . . Each of the Cycle is 3 weeks (21 days).

The response is evaluated by iRECIST. CT scans are repeated every 12 weeks to confirm CR or PD. For example, CT scan images can be reviewed by blinded independent central review.

### Study Endpoints

Primary endpoints of the study include Overall response rate (ORR; commit to collect CT scans imaging for blinded independent central review).

Secondary end points of this study include ORR (investigator review), Duration of response (DOR), progression-free survival (PFS), and Overall Survival (OS).

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

What is claimed is:

1. A method for treating a cancer in a patient in need thereof in one or more treatment cycles, comprising:
(i) administering Compound I or a pharmaceutically acceptable salt thereof to the patient for a first period of time within each of the one or more treatment cycles; and
(ii) administering a PD-1 inhibitor to the patient after the administration of Compound I or a pharmaceutically acceptable salt thereof,
wherein Compound I has the structure of:

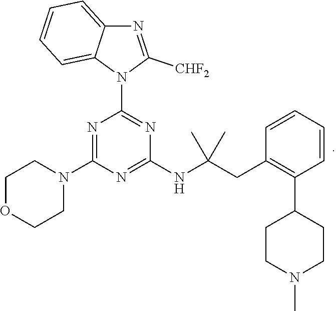 [SMILES: CN1CCC(c2ccccc2CC(C)(C)Nc2nc(N3CCOCC3)nc(-n3c(C(F)F)nc4ccccc43)n2)CC1]

2. The method of claim 1, wherein each of the one or more treatment cycles is at least about 3, 4, 5, 6, 7, 8, 9, 10, 11, or 12 weeks.

3. The method of claim 1 or 2, wherein each of the one or more treatment cycles is about 6 weeks.

4. The method of claim 1 or 2, wherein each of the one or more treatment cycles is about 3 weeks.

5. The method of claim 1, wherein the PD-1 inhibitor is administered about 5, 6, 7, 8, 9, 10, 11, 12, 13, 14, 15, 16, 17, 18, 19, or 20 days after the first period of time.

6. The method of any one of claims 1-3 and 5, wherein each of the one or more treatment cycles is about 6 weeks and the PD-1 inhibitor is administered about 16 days after the first period of time.

7. The method of any one of claims 1, 2, 4, and 5, wherein each of the one or more treatment cycles is about 3 weeks and the PD-1 inhibitor is administered about 8 days after the first period of time.

8. The method of any one of claims 1 to 7, wherein the first period of time is 3 days, 4 days, 5 days, 6 days, or 1 week.

9. The method of claim 8, wherein the first period of time is 5 days.

10. The method of claim 1, wherein after the first period of time, the PD-1 inhibitor is administered starting on the next day after a second period of time, wherein the second period of time is immediately after the first period.

11. The method of claim 10, wherein the second period of time is about 0, 1, 2, 3, 4, 5, 6, 7, 8, 9, 10, 11, 12, 13, 14, 15, 16, 17, 18, 19, or 20 days.

12. The method of claim 11, wherein the second period of time is about 16 days.

13. The method of claim 11, wherein the second period of time is about 8 days.

14. The method of any one of claims 1-13, wherein Compound I or a pharmaceutically acceptable salt thereof is administered daily in the first period of time.

15. The method of claim 1, wherein Compound I or a pharmaceutically acceptable salt thereof is administered to the patient on an intermittent dosing schedule.

16. The method of claim 15, wherein Compound I or a pharmaceutically acceptable salt thereof is only administered for the first period of time of each of the one or more treatment cycles and not on the days after the first period of time, and the first period of time is shorter than each of the one or more treatment cycles.

17. The method of claim 15 or 16, wherein the first period of time is 3 days, 4 days, 5 days, 6 days, or 1 week.

18. The method of any one of claims 15 to 17, wherein the first period of time is 5 days.

19. The method of any one of claims 1 to 18, wherein Compound I or a pharmaceutically acceptable salt thereof is administered once a day at a dosage of 60 mg.

20. The method of any one of claims 1 to 19, wherein Compound I or a pharmaceutically acceptable salt thereof is administered orally.

21. The method of any one of claims 1 to 20, wherein the PD-1 inhibitor is administered once, twice, or three times in each of the one or more treatment cycles.

22. The method of claim 21, wherein the PD-1 inhibitor is administered once in each of the one or more treatment cycles.

23. The method of claim 22, wherein when each of the one or more treatment cycles is about 6 weeks, the PD-1 inhibitor is administered on Day 22 of each of the one or more treatment cycles.

24. The method of claim 22, wherein when each of the one or more treatment cycles is about 3 weeks, the PD-1 inhibitor is administered on Day 14 of each of the one or more treatment cycles.

25. The method of claim 21, wherein the PD-1 inhibitor is administered twice in each of the one or more treatment cycles.

26. The method of claim 25, wherein when each of the one or more treatment cycles is about 6 weeks, the PD-1 inhibitor is administered on Day 14 and Day 35 of each of the one or more treatment cycles.

27. The method of any one of claims 1 to 26, wherein the PD-1 inhibitor is pembrolizumab, nivolumab, cemiplimab, or dostarlimab, or a variant or biosimilar thereof, or combinations thereof.

28. The method of claim 27, wherein the PD-1 inhibitor is pembrolizumab, or a variant or biosimilar thereof.

29. The method of any one of claims 1 to 28, wherein the PD-1 inhibitor is administered for at least 6 weeks.

30. The method of any one of claims 1 to 29, wherein the PD-1 inhibitor is administered for 6, 9, 12, 15, 18, 21, 24, 27, or 30 weeks.

31. The method of any one of claims 1 to 30, wherein the PD-1 inhibitor is administered once every 3 weeks or once every 6 weeks.

32. The method of any one of claims 1 to 31, wherein the PD-1 inhibitor is administered at a dosage of 200 mg or 400 mg.

33. The method of claim 31, wherein the PD-1 inhibitor is administered once every 6 weeks.

34. The method of claim 32, wherein the PD-1 inhibitor is administered at a dosage of 400 mg.

35. The method of claim 31, wherein the PD-1 inhibitor is administered once every 3 weeks.

36. The method of claim 34, wherein the PD-1 inhibitor is administered at a dosage of 200 mg.

37. The method of any one of claims 1-36, wherein the PD-1 inhibitor is administered intravenously.

38. The method of any one of claims 1 to 37, wherein the first period starts on Day 1, Day 2, Day 3, Day 4, Day 5, Day 6, Day 7, or Day 8 of each of the one or more treatment cycles.

39. The method of any one of claims 1 to 38, wherein the first period starts on Day 1 of each of the one or more treatment cycles.

40. The method of claim 1, wherein each of the one or more treatment cycles is 6 weeks, Compound I or a pharmaceutically acceptable salt thereof is administered on Days 1 to 5, and then the PD-1 inhibitor is administered once on Day 22.

41. The method of claim 1, wherein each of the one or more treatment cycles is 3 weeks, Compound I or a pharmaceutically acceptable salt thereof is administered on Days 1 to 5, and then the PD-1 inhibitor is administered once on Day 14.

42. The method of any one of claims 1 to 41, wherein the cancer is lung cancer (e.g., non-small cell lung cancer), melanoma, renal cell cancer, head and neck cancer, breast cancer (e.g., triple-negative breast cancer), non-Hodgkin's lymphoma, Hodgkin lymphoma, colorectal cancer, urothelial carcinoma, gastric cancer, cervical cancer, hepatocellular carcinoma, Merkel cell carcinoma, esophageal cancer, endometrial cancer, cutaneous squamous cell carcinoma, or mesothelioma.

43. The method of claim 42, wherein the cancer is lung cancer.

44. The method of claim 43, wherein the lung cancer is non-small cell lung cancer.

45. The method of claim 42, wherein the cancer is melanoma.

46. The method of claim 42, wherein the cancer is renal cell cancer.

47. The method of claim 42, wherein the cancer is hepatocellular carcinoma.

48. The method of claim 42, wherein the cancer is colorectal cancer.

49. The method of claim 48, wherein the colorectal cancer is microsatellite instability-high or mismatch repair deficient colorectal cancer.

50. The method of any one of claims 1 to 49, further comprising (iii) evaluating efficacy of the administration of the combination of Compound I or a pharmaceutically acceptable salt thereof and the PD-1 inhibitor by measuring levels of regulatory T-cells (CD25hi FoxP3+ T cells) or effector T cells (e.g., CD8+ and CD4+ cells).

51. The method of claim 50, wherein a level of regulatory T-cells (CD25hi FoxP3+ T cells) or effector T cells (e.g., CD8+ and CD4+ cells) that is less than or equal to about 70%, 65%, 60%, 55%, 50%, 45%, 40%, 35%, 30%, 25%, 20%, 15%, or 10% of a baseline level of the regulatory T-cells (CD25hi FoxP3+ T cells) or effector T cells (e.g., CD8+ and CD4+ cells) indicates that the one or more treatment cycles of administration of the administration of the combination of Compound I or a pharmaceutically acceptable salt thereof and the PD-1 inhibitor is effective.

52. The method of claim 50 or 51, wherein a level of regulatory T-cells (CD25hi FoxP3+ T cells) or effector T cells (e.g., CD8+ and CD4+ cells) that is less than or equal to about 65% of a baseline level of the regulatory T-cells (CD25hi FoxP3+ T cells) or effector T cells (e.g., CD8+ and CD4+ cells) indicates that the one or more treatment cycles of administration of the administration of the combination of Compound I or a pharmaceutically acceptable salt thereof and the PD-1 inhibitor is effective.

53. The method of any one of claims 1 to 52, wherein the co-administration of Compound I or pharmaceutically acceptable salt thereof and the PD-1 inhibitor is effective in treating the cancer.

54. A method for treating a cancer in a patient in need thereof in one or more treatment cycles, comprising:
(i) administering Compound I, or a pharmaceutically acceptable salt thereof, to the patient on Days 1 to 5 of each of the one or more treatment cycles; and
(ii) administering a PD-1 inhibitor to the patient on Day 22 of each of the treatment cycles;
wherein each of the one or more treatment cycles is 6 weeks; and
wherein Compound I has the structure of:

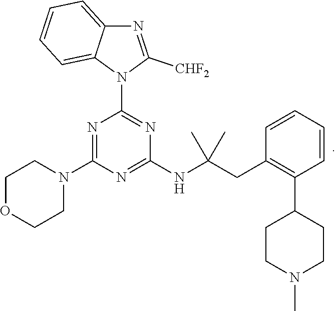 [SMILES: CN1CCC(c2ccccc2CC(C)(C)Nc2nc(N3CCOCC3)nc(-n3c(C(F)F)nc4ccccc43)n2)CC1]

55. A method for treating a cancer in a patient in need thereof in one or more treatment cycles, comprising:
(i) administering Compound I or a pharmaceutically acceptable salt thereof to the patient on Days 1 to 5 of each of the one or more treatment cycles; and
(ii) administering a PD-1 inhibitor to the patient on Day 14 of each of the treatment cycles;
wherein each of the one or more treatment cycles is 3 weeks; and
wherein Compound I has the structure of:

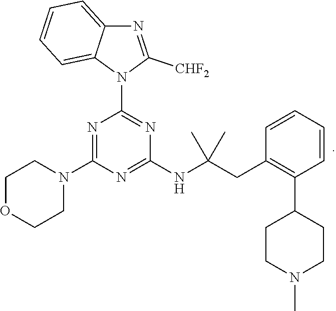 [SMILES: CN1CCC(c2ccccc2CC(C)(C)Nc2nc(N3CCOCC3)nc(-n3c(C(F)F)nc4ccccc43)n2)CC1]

56. A method for treating a cancer in a patient in need thereof in one or more treatment cycles, comprising:
(i) administering Compound I or a pharmaceutically acceptable salt thereof to the patient in Week 1 of odd cycles (e.g., Cycle 1) of the one or more treatment cycles; and
(ii) administering a PD-1 inhibitor to the patient 1 in Week 1 of even cycles (e.g., Cycle 2) of each of the treatment cycles;
wherein each of the one or more treatment cycles is 3, 6, or 9 weeks; and
wherein Compound I has the structure of:

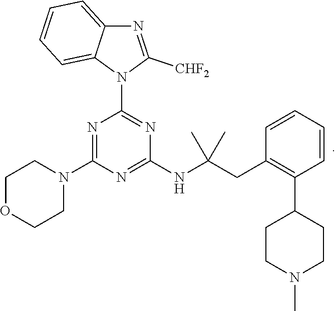 [SMILES: CN1CCC(c2ccccc2CC(C)(C)Nc2nc(N3CCOCC3)nc(-n3c(C(F)F)nc4ccccc43)n2)CC1]

57. A method for treating a cancer in a patient in need thereof in one or more treatment cycles, comprising:
(i) administering Compound I or a pharmaceutically acceptable salt thereof to the patient in Week 1 of each of the one or more treatment cycles; and
(ii) administering a PD-1 inhibitor to the patient in Week 2 of each of the treatment cycles;
wherein each of the one or more treatment cycles is 3 weeks; and
wherein Compound I has the structure of:

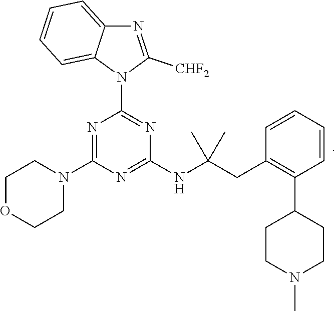 [SMILES: CN1CCC(c2ccccc2CC(C)(C)Nc2nc(N3CCOCC3)nc(-n3c(C(F)F)nc4ccccc43)n2)CC1]

58. A method for treating a cancer in a patient in need thereof, comprising:
(i) administering Compound I or a pharmaceutically acceptable salt thereof to the patient for 1, 2, 3, 4, or 5 days once daily in every 3, 6, or 9 weeks; and
(ii) administering a PD-1 inhibitor to the patient once every 3 or 6 weeks;
wherein Compound I has the structure of:

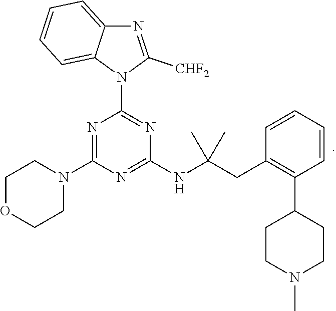 [SMILES: CN1CCC(c2ccccc2CC(C)(C)Nc2nc(N3CCOCC3)nc(-n3c(C(F)F)nc4ccccc43)n2)CC1]

59. A pharmaceutical composition comprising Compound I, having the structure: 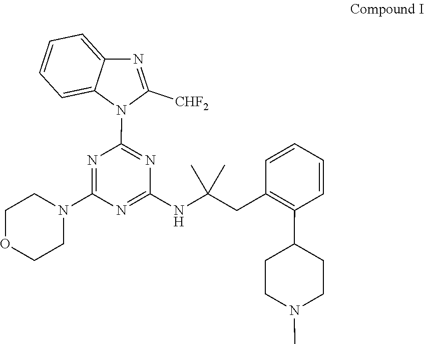 [SMILES: CN1CCC(c2ccccc2CC(C)(C)Nc2nc(N3CCOCC3)nc(-n3c(C(F)F)nc4ccccc43)n2)CC1]
or a pharmaceutically acceptable salt thereof, for use in treating cancer in a patient in combination with a PD-1 inhibitor, wherein
(i) Compound I or a pharmaceutically acceptable salt thereof is administered to the patient for a first period of time within each of the one or more treatment cycles; and
(ii) the PD-1 inhibitor is administered to the patient after the administration of Compound I or a pharmaceutically acceptable salt thereof.
